(19) United States
(12) Patent Application Publication
(10) Pub. No.: US 2020/0054683 A1
(43) Pub. Date: Feb. 20, 2020

(54) METHODS OF TREATING GRAFT VERSUS HOST DISEASE (GVHD) OR EPIDERMOLYSIS BULLOSA (EB) WITH EXOSOMES

(71) Applicant: Agency for Science, Technology and Research (A*STAR), Singapore (SG)

(72) Inventor: Sai Kiang Lim, Singapore (SG)

(73) Assignee: Agency for Science, Technology and Research (A*STAR), Singapore (SG)

(21) Appl. No.: 16/661,657

(22) Filed: Oct. 23, 2019

Related U.S. Application Data
(62) Division of application No. 15/509,072, filed on Mar. 6, 2017.

(30) Foreign Application Priority Data
Sep. 15, 2014 (SG) PCT/SG2014/000434

(51) Int. Cl. A61K 35/28; A61K 39/00

(52) U.S. Cl. CPC A61K 35/28; A61K 39/0008; A61K 39/001

(57) ABSTRACT

We describe the use of exosome such as mesenchymal stem cell exosomes in a method of promoting, restoring or enhancing homeostasis in an individual suffering from graft versus host disease (GVHD) or epidermolysis bullosa (EB). The homeostasis may comprise immune homeostasis such as maintenance of an immune response. The method may comprise administering a therapeutically effective amount of exosome to the individual.

### CROSS REFERENCE TO RELATED APPLICATIONS

This application is a divisional under 35 U.S.C. § 121 of co-pending U.S. application Ser. No. 15/509,072 filed Mar. 6, 2017, which is a National Phase Entry application under 35 U.S.C. § 371 of International Application No. PCT/SG2015/000083 filed Mar. 19, 2015, which claims benefit of International Application No. PCT/SG2014/000434, filed Sep. 15, 2014, the contents of each of which are incorporated herein by reference in their entireties.

### FIELD

The present invention relates to the fields of medicine, cell biology, molecular biology and genetics. This invention also relates to the field of medicine.

### BACKGROUND

Homeostasis is essentially a state of equilibrium and differs from a chemical equilibrium which is determined by the relative concentration of the substrate and product in a microenvironment with defined physical parameters e.g. temperature, pressure. In homeostasis, the equilibrium is defined for each living system and is the point most conducive for the viability of the organism. It is also the state that various biological systems work to achieve and maintain.

Homeostasis could refer to the stable internal biological microenvironment within an organism, system (e.g. cardiovascular, immune), organ, tissues or cells. The microenvironment could include the number, state and type of cells, composition of the extracellular matrix, biochemical and biophysical parameters. Living systems function optimally only within narrowly defined microenvironments. Deviation from a defined microenvironment or loss of homeostasis results in sub-optimal functions that could lead to disease or tissue injury.

Conversely, disease or tissue injury disrupts homeostasis, and repair and recovery involve a return to homeostasis. For example, the physiological pH of blood is 7.4 and the blood buffering system has evolved to equilibrate at pH 7.4 to maintain a homeostatic pH of 7.4. This buffering system has the capacity to buffer the homeostatic pH against changes caused by external factors. When this capacity is exceeded or exhausted, the physiological pH cannot be maintained and there is a loss of pH homeostasis resulting in either acidosis or alkalosis with dire consequences.

Another example is immune homeostasis. Regulating immune homeostasis is fundamental to the body's defence against pathogens and diseased tissues, and minimization of collateral tissue damages. Dysregulation of immune homeostasis compromises the body's defence and self versus non self recognition leading to disease or autoimmunity. Hence restoring or maintaining homeostasis is important not only in maintaining the health or well being of the organism, but also in repair and recovery from disease and injury.

The major players in the regulation or maintenance of homeostasis are signaling molecules such as cytokines, chemokines, growth factors hormones, their receptors and the enzymes which translate the signals into physiological outcomes. The activity of these hormones and their receptors is highly regulated by positive and negative feedback systems that are sensitive to the microenvironment. This sensitivity to the microenvironment, and the positive and negative feedback systems are underpinned primarily by enzymes.

Enzymes are essentially the workhorses for all biological activities from gene replication and transcription to communication, metabolism and cell death. Enzymes have attributes that make them important contributors to homeostasis. Enzyme activity is dependent on relative concentration of substrate, product and co-factors, and external factors such as temperature, pH and tissue or cellular architecture i.e. enzymes are sensitive to their biochemical and biophysical microenvironment and their equilibrium state is evolutionarily calibrated to the homeostatic state.

Deviation from the equilibrium state will inhibit, favour or reverse the reaction until equilibrium is restored. Unlike catalysts, most enzymes particularly the rate-limiting enzymes have to be activated and their activity is highly regulated by feedback mechanisms to maintain a physiological equilibrium rather than a stoichiometric equilibrium.

### SUMMARY

We now demonstrate the capacity of exosomes such as mesenchymal stem cell exosomes to restore homeostasis such as cell and immune homeostasis.

Exosomes such as mesenchymal stem cell exosomes are therefore particularly useful in the treatment or prevention of diseases in which homeostasis is disrupted or otherwise abnormal, such as graft versus host disease (GVHD) or epidermolysis bullosa (EB).

According to a 1^{st} aspect of the present invention, we provide an exosome for use in a method of promoting, restoring or enhancing homeostasis in an individual suffering from graft versus host disease (GVHD) or epidermolysis bullosa (EB).

The homeostasis may comprise immune homeostasis. The homeostasis may comprise maintenance of an immune response.

The method may comprise administering a therapeutically effective amount of exosome to the individual.

The disease may comprise graft versus host disease (GVHD). The disease may comprise acute graft versus host disease (aGVHD). The disease may comprise chronic graft versus host disease (cGVHD). The disease may comprise transfusion-associated graft versus host disease.

The disease may comprise epidermolysis bullosa (EB). The disease may comprise epidermolysis bullosa simplex. The disease may comprise junctional epidermolysis bullosa. The disease may comprise dystrophic epidermolysis bullosa. The disease may comprise lethal acantholytic epidermolysis bullosa. The disease may comprise epidermolysis bullosa acquisita.

The exosome may comprise a mesenchymal stem cell exosome. The exosome may comprise at least one biological property of a mesenchymal stem cell. The exosome may comprise a biological activity of a mesenchymal stem cell conditioned medium (MSC-CM).

The exosome may comprise cardioprotective activity. The exosome may be capable of reducing infarct size. The exosome may be capable of reducing infarct size as assayed in a mouse or pig model of myocardial ischemia and reperfusion injury.

The exosome may be capable of reducing oxidative stress. The exosome may be capable of reducing oxidative stress as assayed in an in vitro assay of hydrogen peroxide (H₂O₂)-induced cell death.

The exosome may have a size of between 50 nm and 100 nm as determined by electron microscopy

There is provided, according to a 2^{nd} aspect of the present invention, an exosome for use in a method of treatment or prevention of a disease characterised by an alteration of homeostasis. The disease may comprise graft versus host disease (GVHD). The disease may comprise epidermolysis bullosa (EB).

We provide, according to a 3^{rd} aspect of the present invention, use of an exosome in the preparation of a medicament for promoting, restoring or enhancing homeostasis in an individual suffering from graft versus host disease (GVHD) or epidermolysis bullosa (EB).

As a 4^{th} aspect of the present invention, there is provided the use of an exosome in the preparation of a medicament for the treatment or prevention of a disease characterised by an alteration of homeostasis, in which the disease comprises graft versus host disease (GVHD) or epidermolysis bullosa (EB).

We provide, according to a 5^{th} aspect of the present invention, a method of treatment of an individual suffering from graft versus host disease (GVHD) or epidermolysis bullosa (EB), the method comprising administering an exosome, preferably a mesenchymal stem cell exosome, to the individual.

The practice of the present invention will employ, unless otherwise indicated, conventional techniques of chemistry, molecular biology, microbiology, recombinant DNA and immunology, which are within the capabilities of a person of ordinary skill in the art. Such techniques are explained in the literature. See, for example, J. Sambrook, E. F. Fritsch, and T. Maniatis, 1989, Molecular Cloning: A Laboratory Manual, Second Edition, Books 1-3, Cold Spring Harbor Laboratory Press; Ausubel, F. M. et al. (1995 and periodic supplements; Current Protocols in Molecular Biology, ch. 9, 13, and 16, John Wiley & Sons, New York, N.Y.); B. Roe, J. Crabtree, and A. Kahn, 1996, DNA Isolation and Sequencing: Essential Techniques, John Wiley & Sons; J. M. Polak and James O'D. McGee, 1990, In Situ Hybridization: Principles and Practice; Oxford University Press; M. J. Gait (Editor), 1984, Oligonucleotide Synthesis: A Practical Approach, Irl Press; D. M. J. Lilley and J. E. Dahlberg, 1992, Methods of Enzymology: DNA Structure Part A: Synthesis and Physical Analysis of DNA Methods in Enzymology, Academic Press; Using Antibodies: A Laboratory Manual: Portable Protocol NO. I by Edward Harlow, David Lane, Ed Harlow (1999, Cold Spring Harbor Laboratory Press, ISBN 0-87969-544-7); Antibodies: A Laboratory Manual by Ed Harlow (Editor), David Lane (Editor) (1988, Cold Spring Harbor Laboratory Press, ISBN 0-87969-314-2), 1855. Handbook of Drug Screening, edited by Ramakrishna Seethala, Prabhavathi B. Fernandes (2001, New York, N.Y., Marcel Dekker, ISBN 0-8247-0562-9); and Lab Ref: A Handbook of Recipes, Reagents, and Other Reference Tools for Use at the Bench, Edited Jane Roskams and Linda Rodgers, 2002, Cold Spring Harbor Laboratory, ISBN 0-87969-630-3. Each of these general texts is herein incorporated by reference.

### BRIEF DESCRIPTION OF THE FIGURES

FIG. 1 is a drawing showing the increasing induction of ERK1/2 phosphorylation by exosomes with increasing AMP concentration.

FIG. 2 is a drawing showing induction of ERK1/2 phosphorylation under non-limiting AMP concentration was not affected by increasing concentration of exosome.

FIG. 3A is a drawing showing the mean rejection score of allogeneic skin graft in saline- and exosome-treated mice.

FIG. 3B is a drawing showing representative skin grafts in saline- and exosome-treated mice.

FIG. 3C is a drawing showing exosome-induced elevated Treg level in graft recipients but not ungrafted animals.

FIG. 3D is a drawing showing exosomes have no effect on Treg level in animals that were not immunologically challenged.

FIG. 4 is a drawing showing cell adhesion after 2 hours and 24 hours of single cell suspension plated on to tissue culture plates coated with 6.25, 25 and 200 μg/ml MSC exosomes or 1 mg/ml gelatine solution.

FIG. 5 is a drawing showing grip strength measurements (kg) averaged by treatment group. Error bars represent standard error of the mean (SEM).

FIG. 6A is a drawing showing Western blot analysis of cell or exosome lysates for collagen 7. Lanes from left to right: Molecular weight markers, RDEB human dermal fibroblasts over-expressing collagen 7, RDEB human dermal fibroblasts, normal human dermal fibroblasts and human mesenchymal stem cell (MSC) exosome. The fibroblast lysate was prepared by removing the culture medium from a confluent dish of primary human dermal fibroblasts and adding a cell lysate buffer to the dish. MSC exosomes were prepared as previously described (reference: Lai R C, Arslan F, Lee M M, Sze N S, Choo A, Chen T S, et al. Exosome secreted by MSC reduces myocardial ischemia/reperfusion injury. Stem Cell Res. 2010; 4: 214-22). The fibroblast and exosome lysates were analysed by a standard immunoblotting assay using an Anti-Collagen VII antibody [LH7.2] (ab6312) from Abcam. * indicates collagen 7.

FIG. 6B is a drawing estimating the concentration of collagen 7 in MSC exosomes relative to that in normal human dermal fibroblast. A two-fold serial dilution of MSC exosome lysate starting with 20 μg protein was prepared and loaded in parallel with 200 μg fibroblast lysate. The blot was also probed for CD81 as a loading control.

FIG. 7 is a drawing showing a survival curve of exosome-treated delayed lymphocyte infusion (DLI) mice (model of graft versus host disease).

FIG. 8 is a drawing showing a survival curve of exosome-treated hypomorphic mouse model of dystrophic epidermolysis bullosa.

### DETAILED DESCRIPTION

This disclosure describes the use of MSC exosomes to restore homeostasis in a disease process or injury, and enhance tissue repair and regeneration.

The exosomes may be used to restore homeostasis in a disease in which homeostasis is not normal. The homeostasis may be disrupted, affected or defective etc. in a patient suffering from the disease.

Examples of diseases in which abnormal homeostasis manifests include graft versus host disease (GVHD) and epidermolysis bullosa (EB).

Accordingly, we provide for the use of exosomes, such as those derived from mesenchymal stem cells in the treatment or prevention of graft versus host disease (GVHD) and epidermolysis bullosa (EB).

We have previously demonstrated that MSC exosomes have a diverse and biochemically active proteome (Reference 1, Reference 2, Reference 3, Reference 4, Reference 5, Reference 6, International Patent Publication WO 2012/108842). Many of the proteins are housekeeping enzymes such as the glycolytic enzymes, proteasome etc, and have the capacity to restore homeostasis in many systems, tissues etc.

Accordingly, exosomes, such as mesenchymal stem cell exosomes, may be used to promote, restore or enhance homeostasis. Exosomes may for example be used to promote, restore or enhance cell homeostasis immune homeostasis in a biological environment in need of such, for example in a patient suffering from graft versus host disease (GVHD) or epidermolysis bullosa (EB).

Exosomes, such as mesenchymal stem cell exosomes, may also be used to alleviate, treat or prevent any disease associated with abnormal homeostasis or a defect in homeostasis.

Exosomes, such as mesenchymal stem cell exosomes, may also be used to alleviate, treat or prevent Duchenne muscular dystrophy (DMD), graft versus host disease (GVHD) or epidermolysis bullosa (EB).

### Restoring Cell Homeostasis

During development, growth, injury or disease, cells are either rapidly proliferating or dying. Both processes are important in generating new tissues, tissue remodeling, removal of diseased or injured tissues and tissue repair or regeneration.

The relative rate of these two processes must be carefully regulated to maintain cell homeostasis to ensure sufficient cell number and an appropriate mix of cells in each tissue and organ for normal function. This homeostasis is transiently disrupted during stress or trauma through proliferation or cell death of specific cell types to mount an appropriate defence and attack, and protect the organism from further injury. A prolonged disruption is likely to compromise normal biological activities in the organism leading to deficiencies in tissue repair and recovery.

Therefore, a speedy recovery of cell homeostasis upon resolution of stress or trauma is an important requisite for repair and recovery. Fortunately, cell homeostasis like most biological systems, have positive and negative feedback mechanisms to calibrate the disruption according to the degree of injury and restore homeostasis upon resolution of the injury.

We demonstrate here that mesenchymal stem cell exosomes have the capacity to enhance a more calibrated disruption and restoration of cell homeostasis, specifically in calibrating apoptosis and proliferation to the degree of injury.

We show that mesenchymal stem cell exosomes may be used to treat or prevent graft versus host disease (GVHD).

We also demonstrate that mesenchymal stem cell exosomes may be used to treat or prevent epidermolysis bullosa (EB).

A classic danger signal issued by tissue in distress is extracellular ATP (reviewed in Reference 7, Reference 8). Extracellular ATP stimulates immunogenic cell death (Reference 9, Reference 10) and removes injured and dying cells. During tissue trauma (e.g. shear or mechanical stress (Reference 11, Reference 12), chemotherapy (Reference 9) or hypoxia (Reference 13)), injured and dying cells released ATPs and ADPs into the extracellular space to remove injured and dying cells. Although the effect of extracellular ATP and ADP is severely limited by their short half-lives of less than 1 s (Reference 14) and 3.2 minutes (Reference 15) respectively, sustained injury could lead to accumulation of extracellular ATP, and result in excessive ATP signal-induced cell death and a “bystander effect” on healthy neighbouring cells.

Extracellular ATPs are rapidly degraded by several ecto-enzymes into AMP which is then degraded to adenosine. The only enzyme known to hydrolyse extracellular AMP to adenosine is CD73 (BC065937.1), an ecto 5′nucleotidase (Reference 16). Enzymatically active CD73 is present on the surface of MSC exosome (Reference 16). Since adenosine activates phosphorylation of pro-survival protein kinases such as Akt and Erk1/2 to induce anti-apoptotic effects and has been shown to be protective against myocardial injury (Reference 17, Reference 18), MSC exosomes have the potential to convert a pro-death ATP molecule through AMP to pro-survival adenosine molecule.

### Cell Adhesion Homeostasis

The structural integrity, organization and patterning of multicellular tissues and organisms are dependent on adhesion between cells, and between cell and extracellular matrix (ECM). These adhesions are mediated by cell adhesion molecules (CAMs).

CAMs are mainly glycoproteins located at the cell surface. They mediate cell adhesion by forming complexes and junctions with other CAMs or ECM components to join cells to cells, cells to ECM and ECM to the cell cytoskeleton. Cell adhesion provides the necessary structural support to position and organize cells within a multi-cellular tissue, organ or organism. This structural organization and integrity of a multi-cellular entity is essentially an ordered conduit through which biochemical and biophysical signals for tissue function, morphogenesis, differentiation, repair and homeostasis are transmitted (Frantz, Stewart et al. 2010).

However, the structural integrity of a multi-cellular tissue, organ or organism is not static. To accommodate the highly dynamic flux of activities in a living organism, cell adhesiveness must be coordinately regulated both spatially and temporally to enable appropriate cell migration, changes in cell shape and re-positioning.

Such regulation inevitably disrupts cell adhesion homeostasis, and restoring or maintaining cell adhesion homeostasis is critical to the normal function of multicellular tissues or organisms. Failure to so do either through genetic or injury-induced defects in ECM or CAMs would severely compromise the structural integrity, organization and patterning of multicellular tissue, and consequently its normal functions.

Extracellular Matrix (ECM)

The ECM is essentially a complex cellular product secreted by cells. It consists essentially of water, proteins and polysaccharides. The exact composition and topology of the ECM are tissue-specific and highly sensitive to changes in the microenvironment. The ECM exists in two basic structures: a basement membrane that provides anchoring support to epithelial and endothelial cells or an interstitial matrix that fills space between cells. Both structures have a collagen scaffold with varying proportion of elastin and fibrillin that binds adhesive glycoproteins and proteoglycans.

The ECM through interaction with CAMs that are present on cells provides structural support for the cells. It also acts as a physical buffer to protect cells against micro-environmental changes or insults. More importantly, ECM components bind growth factors and signal receptors, and play key roles in signalling (Kim, Turnbull et al. 2011). The main ECM components are glycosaminoglycans (GAGs) and glycoproteins

The most common GAGs are heparan sulphate, chondroitin sulphate keratan sulphate and hyaluronic acid. Other than hyaluronic acid, most GAGs are usually attached to ECM proteins or cell surface proteins to form proteoglycans. Heparan sulphate is a linear polysaccharide that binds proteins to form proteoglycans. In the ECM, heparan sulphate binds mainly the basement membranes and the multi-domain proteins such as perlecan, agrin, and collagen XVIII.

Chondroitin sulfates are important in tissues with high tensile strength such as cartilage, tendons, ligaments, and walls of the aorta while keratin sulphates are found in cornea, cartilage, bones. Hyaluronic acid is one of the most hydrophilic molecules known in nature and its main function is to regulate tissue hydration and water transport. It is found in most tissues and is most abundant in liquid connective tissues such as joint synovial and eye vitreous fluid.

The major glycoproteins in the ECM consist of two functional types, structural (collagen, elastin) and adhesive (e.g. fibronectin, laminin, vitronectin, thrombospondin etc.).

Collagens are the most abundant protein in our body and are also most abundant in ECM (Patino, Neiders et al. 2002; Ricard-Blum 2011). The collagen superfamily has 28 types of collagens encoded by 43 genes (Chan, Poon et al. 2008). The diversity of this superfamily is further expanded by the use of alternative promoters or splicing and post-translational modifications. The tissue distribution of collagen types is equally diverse with some collagens such as Type I being ubiquitously distributed while type VII is restricted to the basement membrane of the skin. Collagens are structural proteins that provide mechanical support to tissue organization and shape. They also exert biological effects on cellular functions such as cell proliferation, migration, and differentiation through their interaction with cell receptors. As some collagens have restricted tissue distribution, these biological effects are tissue specific.

Elastin, unlike collagens which provide mechanical support, impart elasticity to tissue to enable stretch and return to their original state. They are found in high abundance in blood vessels, the lungs, skin, and ligaments. Elastin is encoded by ELN gene and is deposited as tropoelastin on fibrillin fibrils to form elastic fibres that is capable of supporting long-range deformability and passive recoil without energy input (Kielty, Sherratt et al. 2002).

Other non-structural components of the ECM are fibronectins, laminins, fibrinogen, fibrillins, fibulins, tenascins and thrombospondins (Halper and Kjaer 2014). Most of these proteins not only contribute to the structural integrity of the ECM but also modulates cells activities such as cell signaling, cell motility, shape and polarity

Cell Adhesion Molecules (CAMs)

CAMs are glycoproteins that are present on cell membrane. They interact either with other CAMs on adjacent cells or with proteins of the extra-cellular matrix to join cells to cells, cells to ECM or ECM to the cell cytoskeleton: (Edelman and Crossin 1991). In so doing, CAMs help to strengthen tissue structure, facilitate transmission of cell between cells or microenvironmental signals into cells (Cavallaro and Dejana 2011).

There are 5 major CAM families: the cadherin superfamily, the selectins, the immunoglobulin (Ig) superfamily, syndecans and the integrins. CAM interactions with the ECM are important for diverse biological processes including differentiation, morphogenesis, cell growth, proliferation etc.

The cadherins are calcium-dependent transmembrane glycoproteins. adherins are calcium-dependent transmembrane proteins that generally mediate cell-cell adhesion or cell-cell recognition. They have two consecutive cadherin repeats, with conserved calcium-binding amino acid residues (http://www.genenames.org/genefamily/cdhsf.php). The cadherin superfamily can be phylogenetically divided into three major families: the major cadherin family, the protocadherin family and the cadherin-related family. (http://www.genenames.org/genefamily/cdhsfphp). Members of this family are N-cadherin, P-cadherin, E cadherin and L-CAM.

The integrins are also transmembrane glycoproteins that unlike the immunoglobulin and cadherin superfamilies, bind both ECM molecules and other cell surface proteins. They also link the cytoskeleton and ECM proteins.

Syndecans are transmembrane heparan sulfate proteoglycans (HSPGs) that can also bind to a wide variety of intra- and extra-cellular proteins and growth factor (Tkachenko, Rhodes et al. 2005). Whereas integrins interact primarily with ECM molecules while selectins and other CAMs participate in cell-cell interactions, syndecans could interact with ECM, cell surface molecules and soluble ligands.

The selectins are a family of Ca²⁺-dependent, carbohydrate-binding transmembrane proteins. There are three selectins namely E-selectin in endothelial cells, L-selectin in leukocytes and P-selectin in platelets and endothelial cells. They mediate tissue inflammation distribution and play important roles in many pathophysiological processes.

Ig superfamily of CAMs unlike the other CAMs is calcium independent. They typically bind integrins or other Ig superfamily CAMs. Some examples of CAMS are the intercellular adhesion molecules (ICAMs), vascular-cell adhesion molecule (VCAM-1), platelet-endothelial-cell adhesion molecule (PECAM-1), and neural-cell adhesion molecule (NCAM). The main role of Ig superfamily of CAMs is generally reported to mediate immune and inflammatory responses.

Importance of Cell Adhesion Homeostasis

Cell adhesion is a fundamental physiological function in multicellular organisms and provides the underpinning structural support for the dynamic flux of activities in a living organism.

To effectively support these activities, the state of cell-adhesion must be equally dynamic in its spatial and temporal configuration of ECM and CAM interactions to effect different degrees of adhesiveness necessary for cell migration, changes in cell shape and re-positioning of cells.

However, such dynamic but physiological ECM and CAM interactions would have to be coupled with timely reversion to the homeostatic state of cell adhesion to restore tissue homeostasis and normal tissue functions. Failure to do so will lead to loss of cell adhesion homeostasis that will compromise tissue function.

Diseases Associated with ECMs

The critical importance of ECM and CAMs in multi-cellular tissues and organisms is best evidenced by the numerous severe diseases caused by seemingly minor changes in ECM or CAMs (Jarvelainen, Sainio et al. 2009).

ECM-associated diseases included not only genetic disease but also acquired diseases. The list of diseases include Alzheimer's disease, cardiovascular diseases (e.g. Abdominal aortic aneurysms arterial aneurysms, Atherosclerosis, atherosclerosis and restenosis, Hereditary angiopathy, Hypertension, hypertensive heart disease, Supravalvular aortic stenosis), eye diseases (e.g. autosomal recessively inherited vitreoretinal dystrophy, Cornea plana, Corneal endothelial dystrophy, Congenital corneal stromal dystrophy, Fuchs' endothelial dystrophy Myopia, thyroid-associated ophthalmopathy, Erosive vitreoretinopathy and Wagner disease, Congenital stationary night blindness, keratoconus, Knobloch syndrome, Macular degenerative disease, Stickler syndrome type I, and type II, Weill-Marchesani syndrome), Bone, cartilage and joint diseases (e.g Achondrogenesis, Osteoarthritis, Osteogenesis imperfect, otospondylomegaepiphyseal dysplasia, spondyloepiphyseal dysplasia, Intervertebral disc degeneration, idiopathic osteoporosis, Dyssegmental dysplasia, Kniest dysplasia, Marfan's syndrome, Marshall syndrome, multiple epiphyseal dysplasia, Congenital contractural arachnodactyly, Scheuermann disease, Shprintzen-Goldberg syndrome, Stickler syndrome type I, type II and type III, Synpolydactyly, tendinopathy, pseudoachondroplasia, multiple epiphyseal dysplasia, Weill-Marchesani syndrome) Cancer and cancer progression (e.g. hepatocellular carcinoma, Colon tumorigenesis, gastric cancer, tumorigenesis and metastasis, breast cancer, T-cell leukemia, Prostate cancer), Cutis laxa, Diabetic complications (e.g. nephropathy, retinopathy), Ehlers-Danlos syndrome (classic and vascular), Epidermolysis bullosa (e.g. EB acquisita, Dystrophic EB, Non-Herlitz junctional EB), Fibrotic diseases (chronic kidney diseases, liver fibrosis, Idiopathic pulmonary fibrosis, Cirrhosis), Hepatitis, inflammatory bowel disease, Muscle disorders (e.g. myosclerosis, myopathy, Congenital muscular dystrophy Rhabdomyosarcomas, Schwartz-Jampel syndrome), Lung diseases (e.g. pulmonary emphysema, Bronchiolitis obliterans syndrome), renal disease (e.g. Glomerulopathy, Glomerulosclerosis, Alport syndrome, Collagenofibrotic glomerulopathy), Williams-Beuren syndrome.

CAM-associated diseases include many ECM-associated diseases such as Alzheimer's disease, Multiple Sclerosis and Schizophrenia (Gnanapavan and Giovannoni 2013), atherosclerosis coronary, artery thrombosis, viral myocarditis, cardiomyopathy, reperfusion injury myocardial infarction allograft vasculopathy, inflammatory heart diseases (Jang, Lincoff et al. 1994; Hillis and Flapan 1998; Golias, Tsoutsi et al. 2007), inflammatory diseases e.g. IBD, Crohn's disease, coeliac disease (Mishra, Mishra et al. 1993; Nakamura, Ohtani et al. 1993; Schuermann, Aber-Bishop et al. 1993; Villanacci, Facchetti et al. 1993; Schiller and Elinder 1999), fibrotic skin disorders such as keloids and scleroderma (Halper and Kjaer 2014), cancer and metastasis (Johnson 1991; Johnson 1992) neuroendocrine carcinomas (Deichmann, Kurzen et al. 2003), psoriasis (Cagnoni, Ghersetich et al. 1994), inflammatory myopathies and Duchenne dystrophy (De Bleecker and Engel 1994), multiple sclerosis (Vora, Kidd et al. 1997; Ukkonen, Wu et al. 2007), lung inflammation (Wegner, Gundel et al. 1992; Hellewell 1993).

Therapeutic Strategy to Restore Cell Adhesion Homeostasis

Cell adhesion homeostasis is maintained by a complex network of enzyme and enzyme inhibitors whose activities are dependent on the relative concentration of substrates and products (Lu, Takai et al. 2011).

In reversible enzymatic reactions, disequilibrium in the relative concentration of substrate and product will increase enzymatic activity in the direction favoring equilibrium for that microenvironment. Once equilibrium is reached, enzyme activity ceases.

Therefore, enzymes in the presence of sufficient substrates could mediate homeostasis through their innate capacity to restore equilibrium in the relative concentration of substrates and products. In instances where homeostasis cannot be achieved or is lost through injury or genetic defect, a transient replenishment of key critical substrates or enzymes could potentially restored homeostasis albeit transiently but sufficiently long enough to interrupt a chronic disequilibrium state and provide a window of opportunity for endogenous recovery of homeostasis.

A candidate therapeutic agent that has the potential to restore cell adhesion homeostasis is mesenchymal stem cells (MSCs). MSCs are known to create or modulate stem cell niches that promote the engraftment of stem cells such as hematopoietic stem cells, neural progenitor cells, cardiac stem cells (reviewed (Gotts and Matthay 2012). Stem cell niche is essentially a physically and biologically delineated microenvironment that supports stem cells (Schofield 1978).

A primary function of the niche is to anchor stem cells inside a regulated local microenvironment where variables are calibrated to achieve a stable constant i.e. homeostatic state. As MSC is increasingly reported to exert its biological effects via the secretion of paracrine factors, a logical linear extrapolation would be that MSCs also to modulate the microenvironment of stem cell niches through paracrine factors.

However, no candidate molecules known to be secreted by MSCs could recapitulate the niche modulating effects of MSCs. My group has previously proposed that the active agent in MSC paracrine secretion is exosome and also propose that MSC exosome could restore cell and immune homeostasis.

We provide evidence that MSC exosome could also restore cell adhesion homeostasis. We have previously shown using mass spectrometry that the proteome of MSC exosomes have 4 members of the MMP family, MMP 1, 2, 3 and 10, and 3 tissue inhibitors of MMP, TIMP1, 2 and 3 were present. The proteome of MSC exosome has been reported (International Patent Publication Number WO 2012/108842 and deposited at www.exocarta.com). MMP1 and 2 are gelatinases and MMP3 and 10 are collagenases (Clark, Swingler et al. 2008; Bellayr, Mu et al. 2009).

Their presence and enzymatic activity in MSC exosomes have been confirmed by enzymatic assays and immunoblotting (as described in International Patent Publication Number WO 2012/108842). The presence of TIMP1, 2 and 3 has also been confirmed by immunoblotting. In addition to these, MSC exosomes carried many ECM proteins and CAMs, ECM modifying enzymes (Table DO.

Together, these proteins indicate that MSC exosomes have the potential to restore cell adhesion homeostasis by providing the main substrates and enzymes to complement the residual homeostatic machinery. Homeostasis is achieved when the enzymatic processes within the niche reaches equilibrium.

TABLE D1
List of ECM proteins, CAMs and ECM modifying enzymes that
were detected in the proteome of MSC exosomes (International
Patent Publication Number WO 2012/108842).

| ECM proteins | | | | CAMs | | ECM enzymes |
|---|---|---|---|---|---|---|
| COL7A1 | KRT7 | KRT80 | NID1 | ICAM1 | ITGAV | PLOD2 |
| COL18A1 | FGG | KRT19 | VCAN | CD44 | ITGB1 | ADAM9 |
| COL1A1 | FN1 | KRT27 | SDC1 | CDH13 | ITGB4 | ADAMTS12 |
| COL1A2 | KRT1 | KRT28 | SDC2 | ITGA2 | ITGB5 | ADAMTS12 |
| COL2A1 | KRT2 | KRT72 | SDC4 | CEACAM8 | | ENTPD4 |
| COL3A1 | KRT3 | KRT73 | SPARC | ITGA3 | | MMP1 |
| COL4A2 | KRT4 | KRT74 | TGFBI | CLSTN1 | | MMP1 |
| COL4A3 | KRT5 | KRT76 | THBS1 | CNTN1 | | MMP10 |
| COL6A2 | KRT6C | KRT79 | THBS2 | CTNND1 | | MMP3 |
| ECM1 | KRT8 | KRT84 | TNC | ICAM5 | | PLOD3 |
| COL5A1 | KRT6A | KRT77 | VTN | CTNNA1 | | MMP2 |
| ARMS | FBN1 | KRT15 | | ALCAM | | ADAM10 |
| COL6A1 | KRT6B | KRT78 | | CTNNB1 | | MMP2 |
| AGRN | FBN2 | KRT16 | | FAT | | PCOLCE |
| COL12A1 | FGA | KRT17 | | FAT2 | | PLAU |
| COL14A1 | FGB | KRT18 | | FAT4 | | PLOD1 |
| EFEMP2 | KRT9 | LAMA3 | | ITGA4 | | SERPINE1 |
| FBLN1 | KRT10 | LAMA4 | | ITGA5 | | TIMP1 |
| FLG2 | KRT13 | LAMB1 | | ITGA11 | | TIMP2 |
| FBLN1 | KRT14 | LAMC1 | | ITGAL | | TIMP3 |

### Graft Versus Host Disease (GVHD)

Exosomes, such as mesenchymal stem cell exosomes, may be used to treat or prevent graft versus host disease (GVHD), including any of the types of graft versus host disease set out below.

[The text below is adapted from Graft-versus-host disease. (2015, March 2). In Wikipedia, The Free Encyclopedia. Retrieved 07:46, Mar. 10, 2015, from http://en.wikipedia.org/w/index.php?title=Graft-versus-host_disease&oldid=649503786]

Graft-versus-host disease (GVHD), also known as graft versus host disease, is a common complication following an allogeneic tissue transplant. It is commonly associated with stem cell or bone marrow transplant but the term also applies to other forms of tissue graft. Immune cells (white blood cells) in the tissue (the graft) recognize the recipient (the host) as “foreign”. The transplanted immune cells then attack the host's body cells. GVHD can also occur after a blood transfusion if the blood products used have not been irradiated or treated with an approved pathogen reduction system.

Causes of Graft Versus Host Disease

Under the Billingham Criteria, 3 criteria must be met in order for GVHD to occur (Spiryda, L; Laufer, M R; Soiffer, R J; Antin, J A (2003). “Graft-versus-host disease of the vulva and/or vagina: Diagnosis and treatment”. Biology of Blood and Marrow Transplantation 9 (12): 760-5).
An immuno-competent graft is administered, with viable and functional immune cells.
The recipient is immunologically disparate—histo-incompatible.
The recipient is immuno-compromised and therefore cannot destroy or inactivate the transplanted cells.

After bone marrow transplantation, T cells present in the graft, either as contaminants or intentionally introduced into the host, attack the tissues of the transplant recipient after perceiving host tissues as antigenically foreign. The T cells produce an excess of cytokines, including TNF-α and interferon-gamma (IFNγ). A wide range of host antigens can initiate graft-versus-host-disease, among them the human leukocyte antigens (HLAs). However, graft-versus-host disease can occur even when HLA-identical siblings are the donors. HLA-identical siblings or HLA-identical unrelated donors often have genetically different proteins (called minor histocompatibility antigens) that can be presented by Major histocompatibility complex (MHC) molecules to the donor's T-cells, which see these antigens as foreign and so mount an immune response.

While donor T-cells are undesirable as effector cells of graft-versus-host-disease, they are valuable for engraftment by preventing the recipient's residual immune system from rejecting the bone marrow graft (host-versus-graft). In addition, as bone marrow transplantation is frequently used to treat cancer, mainly leukemias, donor T-cells have proven to have a valuable graft-versus-tumor effect. A great deal of current research on allogeneic bone marrow transplantation involves attempts to separate the undesirable graft-vs-host-disease aspects of T-cell physiology from the desirable graft-versus-tumor effect.

Types of Graft Versus Host Disease

In the clinical setting, graft-versus-host-disease is divided into acute and chronic forms.

The acute or fulminant form of the disease (aGVHD) is normally observed within the first 100 days post-transplant, and is a major challenge to transplants owing to associated morbidity and mortality. (Goker, H; Haznedaroglu, I C; Chao, N J (2001). “Acute graft-vs-host disease Pathobiology and management”. Experimental Hematology 29 (3): 259-77)

The chronic form of graft-versus-host-disease (cGVHD) normally occurs after 100 days. The appearance of moderate to severe cases of cGVHD adversely influences long-term survival (Lee, Stephanie J.; Vogelsang, Georgia; Flowers, Mary E. D. (2003). “Chronic graft-versus-host disease”. Biology of Blood and Marrow Transplantation 9 (4): 215-33)

Clinical Manifestation of Graft Versus Host Disease

In the classical sense, acute graft-versus-host-disease is characterized by selective damage to the liver, skin (rash), mucosa, and the gastrointestinal tract. Newer research indicates that other graft-versus-host-disease target organs include the immune system (the hematopoietic system, e.g., the bone marrow and the thymus) itself, and the lungs in the form of immune-mediated pneumonitis. Biomarkers can be used to identify specific causes of GVHD, such as elafin in the skin (Paczesny, S.; Levine, J. E.; Hogan, J.; Crawford, J.; Braun, T. M.; Wang, H.; Faca, V.; Zhang, Q. et al. (2009). “Elafin is a Biomarker of Graft Versus Host Disease of the Skin”. Biology of Blood and Marrow Transplantation 15 (2): 13-4).

Chronic graft-versus-host-disease also attacks the above organs, but over its long-term course can also cause damage to the connective tissue and exocrine glands.

Acute GVHD of the GI tract can result in severe intestinal inflammation, sloughing of the mucosal membrane, severe diarrhea, abdominal pain, nausea, and vomiting. This is typically diagnosed via intestinal biopsy. Liver GVHD is measured by the bilirubin level in acute patients. Skin GVHD results in a diffuse maculopapular rash, sometimes in a lacy pattern.

Mucosal damage to the vagina can result in severe pain and scarring, and appears in both acute and chronic GVHD. This can result in an inability to have sexual intercourse.[1]

Acute GVHD is staged as follows: overall grade (skin-liver-gut) with each organ staged individually from a low of 1 to a high of 4. Patients with grade IV GVHD usually have a poor prognosis. If the GVHD is severe and requires intense immunosuppression involving steroids and additional agents to get under control, the patient may develop severe infections as a result of the immunosuppression and may die of infection.

In the oral cavity, chronic graft-versus-host-disease manifests as lichen planus with a higher risk of malignant transformation to oral squamous cell carcinoma in comparison to the classical oral lichen planus. Graft-versus-host-disease-associated oral cancer may have more aggressive behavior with poorer prognosis, when compared to oral cancer in non-hematopoietic stem cell transplantation patients (Elad, Sharon; Zadik, Yehuda; Zeevi, Itai; Miyazaki, Akihiro; De Figueiredo, Maria A. Z.; Or, Reuven (2010). “Oral Cancer in Patients After Hematopoietic Stem-Cell Transplantation: Long-Term Follow-Up Suggests an Increased Risk for Recurrence”. Transplantation 90 (11): 1243-4).

Transfusion Associated Graft Versus Host Disease

This type of GVHD is associated with transfusion of un-irradiated blood to immunocompromised recipients. It can also occur in situations in which the blood donor is homozygous and the recipient is heterozygous for an HLA haplotype. It is associated with higher mortality (80-90%) due to involvement of bone marrow lymphoid tissue, however the clinical manifestations are similar to GVHD resulting from bone marrow transplantation. Transfusion-associated GVHD is rare in modern medicine. It is almost entirely preventable by controlled irradiation of blood products to inactivate the white blood cells (including lymphocytes) within (Moroff, G; Leitman, S F; Luban, N L (1997). “Principles of blood irradiation, dose validation, and quality control”. Transfusion 37 (10): 1084-92).

Graft Versus Host Disease in Thymus Transplantation

Thymus transplantation may be said to be able to cause a special type of GVHD because the recipients thymocytes would use the donor thymus cells as models when going through the negative selection to recognize self-antigens, and could therefore still mistake own structures in the rest of the body for being non-self. This is a rather indirect GVHD because it is not directly cells in the graft itself that causes it but cells in the graft that make the recipient's T cells act like donor T cells. It can be seen as a multiple-organ autoimmunity in xenotransplantation experiments of the thymus between different species (Xia, G; Goebels, J; Rutgeerts, O; Vandeputte, M; Waer, M (2001). “Transplantation tolerance and autoimmunity after xenogeneic thymus transplantation”. Journal of immunology 166 (3): 1843-54).

Autoimmune disease is a frequent complication after human allogeneic thymus transplantation, found in 42% of subjects over 1 year post transplantation (Markert, M. Louise; Devlin, Blythe H.; McCarthy, Elizabeth A.; Chinn, Ivan K.; Hale, Laura P. (2008). “Thymus Transplantation”. In Lavini, Corrado; Moran, Cesar A.; Morandi, Uliano et al. Thymus Gland Pathology: Clinical, Diagnostic, and Therapeutic Features. pp. 255-67).

However, this is partially explained by the fact that the indication itself, that is, complete DiGeorge syndrome, increases the risk of autoimmune disease (Markert, M. L.; Devlin, B. H.; Alexieff, M. J.; Li, J.; McCarthy, E. A.; Gupton, S. E.; Chinn, I. K.; Hale, L. P. et al. (2007). “Review of 54 patients with complete DiGeorge anomaly enrolled in protocols for thymus transplantation: Outcome of 44 consecutive transplants”. Blood 109 (10): 4539-47).

Prevention of Graft Versus Host Disease

DNA-based tissue typing allows for more precise HLA matching between donors and transplant patients, which has been proven to reduce the incidence and severity of GVHD and to increase long-term survival (Morishima, Y.; Sasazuki, T; Inoko, H; Juji, T; Akaza, T; Yamamoto, K; Ishikawa, Y; Kato, S et al. (2002). “The clinical significance of human leukocyte antigen (HLA) allele compatibility in patients receiving a marrow transplant from serologically HLA-A, HLA-B, and HLA-DR matched unrelated donors”. Blood 99 (11): 4200-6).

The T-cells of umbilical cord blood (UCB) have an inherent immunological immaturity (Grewal, S. S.; Barker, J N; Davies, S M; Wagner, J E (2003). “Unrelated donor hematopoietic cell transplantation: Marrow or umbilical cord blood?”. Blood 101 (11): 4233-44) and the use of UCB stem cells in unrelated donor transplants has a reduced incidence and severity of GVHD (Laughlin, Mary J.; Barker, Juliet; Bambach, Barbara; Koc, Omer N.; Rizzieri, David A.; Wagner, John E.; Gerson, Stanton L.; Lazarus, Hillard M. et al. (2001). “Hematopoietic Engraftment and Survival in Adult Recipients of Umbilical-Cord Blood from Unrelated Donors”. New England Journal of Medicine 344 (24): 1815-22).

The use of liver-derived hematopoietic stem cells to reconstitute bone marrow has the highest success rate according to recent studies.

Methotrexate, ciclosporin and tacrolimus are common drugs used for GVHD prophylaxis. Graft-versus-host-disease can largely be avoided by performing a T-cell-depleted bone marrow transplant. However, these types of transplants come at a cost of diminished graft-versus-tumor effect, greater risk of engraftment failure, or cancer relapse (Hale, G; Waldmann, H (1994). “Control of graft-versus-host disease and graft rejection by T cell depletion of donor and recipient with Campath-1 antibodies. Results of matched sibling transplants for malignant diseases”. Bone marrow transplantation 13 (5): 597-611) and general immunodeficiency, resulting in a patient more susceptible to viral, bacterial, and fungal infection. In a multi-center study, disease-free survival at 3 years was not different between T cell-depleted and T cell-replete transplants (Wagner, John E; Thompson, John S; Carter, Shelly L; Kernan, Nancy A; Unrelated Donor Marrow Transplantation Trial (2005). “Effect of graft-versus-host disease prophylaxis on 3-year disease-free survival in recipients of unrelated donor bone marrow (T-cell Depletion Trial): A multi-centre, randomised phase trial”. The Lancet 366 (9487): 733-41).

Treatment of Graft Versus Host Disease

Intravenously administered glucocorticoids, such as prednisone, are the standard of care in acute GVHD and chronic GVHD (Menillo, S A; Goldberg, S L; McKiernan, P; Pecora, A L (2001). “Intraoral psoralen ultraviolet a irradiation (PUVA) treatment of refractory oral chronic graft-versus-host disease following allogeneic stem cell transplantation”. Bone Marrow Transplantation 28 (8): 807-8).

The use of these glucocorticoids is designed to suppress the T-cell-mediated immune onslaught on the host tissues; however, in high doses, this immune-suppression raises the risk of infections and cancer relapse. Therefore, it is desirable to taper off the post-transplant high-level steroid doses to lower levels, at which point the appearance of mild GVHD may be welcome, especially in HLA mis-matched patients, as it is typically associated with a graft-versus-tumor effect.

Individuals suffering with ocular surface disease caused by GVHD can often receive PROSE (prosthetic replacement of the ocular surface ecosystem) treatment to reduce symptoms and improve visual functioning. (Takahide K, Parker P M, Wu M, et al. (September 2007). “Use of fluid-ventilated, gas-permeable scleral lens for management of severe keratoconjunctivitis sicca secondary to chronic graft-versus-host disease”. Biology of Blood and Marrow Transplantation 13 (9): 1016-21. doi:10.1016/j.bbmt.2007.05.006. PMC 2168033. PMID 17697963; Jacobs D S, Rosenthal P (December 2007). “Boston scleral lens prosthetic device for treatment of severe dry eye in chronic graft-versus-host disease”. Cornea 26 (10): 1195-9) (prosthetic devices used in PROSE treatment were formerly known as the Boston Scleral Lens and Boston Ocular Surface Prosthesis).

Investigational Therapies for Graft Versus Host Disease

There are a large number of clinical trials either ongoing or recently completed in the investigation of graft-versus-host disease treatment and prevention.

On May 17, 2012, Osiris Therapeutics announced that Canadian health regulators approved Prochymal, its drug for acute graft-versus host disease in children who have failed to respond to steroid treatment. Prochymal is the first stem cell drug to be approved for a systemic disease (World's First Stem-Cell Drug Approval Achieved in Canada, The National Law Review. Drinker Biddle & Reath LLP. 2012-06-12).

### Transfusion-Associated Graft Versus Host Disease

[The text below is adapted from Transfusion-associated graft versus host disease. (2014, March 12). In Wikipedia, The Free Encyclopedia. Retrieved 08:04, Mar. 10, 2015, from http://en.wikipedia.org/w/index.php?title=Transfusion-associated_graft_versus_host_disease&oldid=599310890]

Transfusion-associated graft-versus-host disease (TA-GvHD) is a rare complication of blood transfusion, in which the donor T lymphocytes mount an immune response against the recipient's lymphoid tissue (“Complications of Transfusion: Transfusion Medicine: Merck Manual Professional” at http://www.merckmanuals.com/professional/hematology_and_oncology/transfusion_medicine/complications_of_transfusion.html)

Donor lymphocytes are usually identified as foreign and destroyed by the recipient's immune system. However, in situations where the recipient is immunocompromised (inborn immunodeficiency, acquired immunodeficiency, malignancy), or when the donor is homozygous and the recipient is heterozygous for an HLA haplotype (as can occur in directed donations from first-degree relatives), the recipient's immune system is not able to destroy the donor lymphocytes. This can result in graft-versus-host disease.

Epidemiology and Pathogenesis of Transfusion Associated Graft Versus Host Disease

The incidence of TA-GvHD in immunocompromised patients receiving blood transfusions is estimated to be 0.1-1.0%, and mortality around 80-90%. Mortality is higher in TA-GvHD than in GvHD associated with bone marrow transplantation, where the engrafted lymphoid cells in the bone marrow are of donor origin and therefore the immune reaction is not directed against them.

The most common causes of death in TA-GvHD are infections and hemorrhages secondary to pancytopenia and liver dysfunction.

Presentation and Diagnosis of Transfusion Associated Graft Versus Host Disease

Clinical Manifestations of Transfusion-Associated Graft Versus Host Disease

The clinical presentation is the same as GvHD occurring in other settings, such as bone marrow transplantation. TA-GvHD can develop four to thirty days after the transfusion. Typical symptoms include:
fever
erythematosus maculopapular rash, which can progress to generalised erythroderma
toxic epidermal necrolysis in extreme cases

Other symptoms can include cough, abdominal pain, vomiting, and profuse diarrhea (up to 8 liters/day).

Laboratory Manifestations of Transfusion-Associated Graft Versus Host Disease

Laboratory findings include pancytopenia, abnormal liver enzymes, and electrolyte imbalance (when diarrhea is present).

Diagnosis of Transfusion-Associated Graft Versus Host Disease

TA-GvHD can be suspected from a biopsy of the affected skin, and established by HLA analysis of the circulating lymphocytes. This testing can identify circulating lymphocytes with a different HLA type than the tissue cells of the host.

Treatment and Prevention of Transfusion-Associated Graft Versus Host Disease

Treatment is only supportive, as no available form of therapy has proven effective in treating TA-GvHD.

Prevention includes gamma irradiation of the lymphocyte-containing blood products. This procedure should be performed in transfusions when:
The recipient is immunocompromised.
The blood components are from a family donor.
HLA-matched platelets are transferred.

Another means of prevention is the use of third- or fourth-generation leukoreduction filters, although the efficacy of this procedure has not yet been documented.

### Epidermolysis Bullosa (EB)

Exosomes, such as mesenchymal stem cell exosomes, may be used to treat or prevent epidermolysis bullosa (EB), including any of the types of epidermolysis bullosa set out below.

Epidermolysis bullosa (EB) refers to a group of rare genetic skin disorders characterized by skin disadherence and poor wound epithelialisation (Kopecki et al 2009). It has a wide clinical spectrum of severity that correlates with the underlying genetic disorder.

The main genetic disorders in EB involve primarily the structural proteins that hold together skin layers at the dermal-epidermal junction (DEJ).

The most severe forms of EB are the junctional EBs (JEBs) or the dystrophic EBs (DEB). The genes that are mutated in these EBs have identified and included Laminin-5, Type XVII collagen, integrin α6β4, Type VII collagen (Table D2 below).

TABLE D2
List of genes involved in the main EB types and the affected
gene and proteins (reproduced from Kopecki et al, 2009).

| Main EB | | | |
|---|---|---|---|
| type/subtype | Inheritance | Gene involved | Protein involved |
| EBS | | | |
| Suprabasal EBS | AD | PKP; DSP | Plakoplilin-1; Desmoplaktin |
| Basal EBS | AD | KRT5; KRT14; | Kratin 5 & 14; |
| | | PLEC1; ITGA6; ITGB4 | Plectin; Integrin α6β4 |
| JEB | | | |
| JEB-Herlitz | AR | LAMA3; LAMB3; LAMC2 | Laminin-5 |
| JEB, other | AR | LAMA3; LAMB3; LAMC2; | Laminin-5; Type XVII |
| | | COL17A1; ITGA6; ITGB4 | collagen; Integrin α6β4 |
| DEB | | | |
| Dominant DEB (DDEB) | AD | COL7A1 | Type VII collagen |
| Recessive DEB (RDEB) | AR | COL7A1 | Type VII collagen |
| Kindler Syndrome | | | |
| — | AR | KIND1 | Kindlin-1 |

AD = autosomal dominant
AR = autosomal recessive

Any of the various types of epidermolysis bullosa set out below and in this document may be treated with exosomes according to the methods and compositions described here.

### Epidermolysis Bullosa (EB)

[The text below is adapted from Epidermolysis bullosa. (2014, July 7). In Wikipedia, The Free Encyclopedia. Retrieved 11:50, Sep. 11, 2014, from http://en.wikipedia.org/w/index.php?title=Epidermolysis_bullosa&oldid=615914312]

Epidermolysis bullosa (EB) is an inherited connective tissue disease causing blisters in the skin and mucosal membranes, with an incidence of 1/50,000. It is a result of a defect in anchoring between the epidermis and dermis, resulting in friction and skin fragility. Its severity ranges from mild to lethal.

Classification of Epidermolysis Bullosa

Epidermolysis bullosa refers to a group of inherited disorders that involve the formation of blisters following trivial trauma. Over 300 mutations have been identified in this condition. They have been classified into the following types:
Epidermolysis bullosa simplex
Junctional epidermolysis bullosa
Dystrophic epidermolysis bullosa
Epidermolysis bullosa, lethal acantholytic
Epidermolysis bullosa acquisita

Pathophysiology of Epidermolysis Bullosa

The human skin consists of two layers: an outermost layer called the epidermis and a layer underneath called the dermis. In individuals with healthy skin, there are protein anchors between these two layers that prevent them from moving independently from one another (shearing). In people born with EB, the two skin layers lack the protein anchors that hold them together, resulting in extremely fragile skin—even only minor mechanical friction (like rubbing or pressure) or trauma will separate the layers of the skin and form blisters and painful sores. Sufferers of EB have compared the sores with third-degree burns. Furthermore, as a complication of the chronic skin damage, people suffering from EB have an increased risk of malignancies (cancers) of the skin.

Treatment of Epidermolysis Bullosa

Recent research has focused on changing the mixture of keratins produced in the skin. There are 54 known keratin genes—of which 28 belong to the type I intermediate filament genes and 26 to type II—which work as heterodimers. Many of these genes share substantial structural and functional similarity, but they are specialized to cell type and/or conditions under which they are normally produced. If the balance of production could be shifted away from the mutated, dysfunctional keratin gene toward an intact keratin gene, symptoms could be reduced. For example, sulforaphane, a compound found in broccoli, was found to reduce blistering in a mouse model to the point where affected pups could not be identified visually, when injected into pregnant mice (5 μmol/day=0.9 mg) and applied topically to newborns (1 μmol/day=0.2 mg in jojoba oil).

Current clinical research at the University of Minnesota has included a bone marrow transplant to a 2-year-old child who is one of 2 brothers with EB. The procedure was successful, strongly suggesting that a cure may have been found. A second transplant has also been performed on the child's older brother, and a third transplant is scheduled for a California baby. The clinical trial will ultimately include transplants to 30 subjects. However, the severe immunosuppression that bone marrow transplantation requires causes a significant risk of serious infections in patients with large scale blisters and skin erosions. Indeed, at least four patients have died in the course of either preparation for or institution of bone marrow transplantation for epidermolysis bullosa, out of only a small group of patients treated so far.

Epidemiology of Epidermolysis Bullosa

An estimated 50 in 1 million live births are diagnosed with EB, and 9 in 1 million sufferers are in the general population. Of these cases, approximately 92% are epidermolysis bullosa simplex (EBS), 5% are dystrophic epidermolysis bullosa (DEB), 1% are junctional epidermolysis bullosa (JEB), and 2% are unclassified. Carrier frequency ranges from 1 in 333 for JEB, to 1 in 450 for DEB; the carrier frequency for EBS is presumed to be much higher than JEB or DEB.

The disorder occurs in every racial and ethnic group throughout the world and affects both sexes.

Monitoring of Epidermolysis Bullosa

The Epidermolysis Bullosa Activity and Scarring index (EBDASI) is a scoring system that objectively quantifies the severity of Epidermolysis Bullosa (EB). The EBDASI is a tool for clinicians and patients to monitor the severity of the disease. It has also been designed to evaluate the response to new therapies for the treatment of EB. The EBDASI was developed and validated by Professor Dedee Murrell and her team of students and fellows at the St George Hospital, University of New South Wales, in Sydney, Australia. It was presented at the International Investigative Dermatology congress in Edinburgh in 2013 and a paper-based version was published in the Journal of American Academy of Dermatology

### Epidermolysis Bullosa Simplex

[The text below is adapted from Epidermolysis bullosa simplex. (2014, March 21). In Wikipedia, The Free Encyclopedia. Retrieved 11:54, Sep. 11, 2014, from http://en.wikipedia.org/w/index.php?title=Epidermolysis_bullosa_simplex&oldid=600649962]

Epidermolysis bullosa simplex is a form of epidermolysis bullosa that causes blisters at the site of rubbing. It typically affects the hands and feet, and is typically inherited in an autosomal dominant manner, affecting the keratin genes KRT5 and KRT14.

Classification of Epidermolysis Bullosa Simplex

Epidermolysis bullosa simplex may be divided into multiple types:

Epidermolysis bullosa simplex with migratory circinate erythema is described in OMIM entry number 609352 (locus and gene 12q13 (KRT5)).

Epidermolysis bullosa simplex with mottled pigmentation is described in OMIM entry number 131960 (locus and gene 12q13 (KRT5)). It is associated with a recurrent mutation in KRT14.

Epidermolysis bullosa simplex, autosomal recessive is described in OMIM entry number 601001 (locus and gene 17q12-q21 (KRT14)).

Generalized epidermolysis bullosa simplex is described in OMIM entry number 131900 (locus and gene 17q12-q21 (KRT5), 12q13 (KRT14)). Generalized epidermolysis bullosa simplex is also known as “Koebner variant of generalized epidermolysis bullosa simplex”. It presents at birth to early infancy with a predilection for the hands, feet, and extremities, and palmar-plantar hyperkeratosis and erosions may be present.

Localized epidermolysis bullosa simplex is described in OMIM entry number 131800 (locus and gene 17q12-q21 (KRT5), 17q11-qter, 12q13 (KRT14)). Localized epidermolysis bullosa simplex is also known as “Weber-Cockayne syndrome,” and “Weber-Cockayne variant of generalized epidermolysis bullosa simplex”. It is characterized by onset in childhood or later in life, and is the most common variant of epidermolysis bullosa simplex.

Epidermolysis bullosa herpetiformis is described in OMIM entry number 131760 (locus and gene 17q12-q21 (KRT5), 12q13 (KRT14)). Epidermolysis bullosa herpetiformis is also known as “Dowling-Meara epidermolysis bullosa simplex”. It presents at birth with a generalized distribution, often with oral mucosa involvement and variable lesions in infancy.

Epidermolysis bullosa simplex with muscular dystrophy is described in OMIM entry number 226670 (locus and gene 8q24 (PLEC1)). A rare clinical entity, epidermolysis bullosa simplex is the only epidermolytic epidermolysis bullosa described that is not caused by a keratin mutation, presenting as a generalized intraepidermal blistering similar to the Koebner variant of generalized epidermolysis bullosa simplex, but also associated with adult onset muscular dystrophy.

Epidermolysis bullosa simplex with pyloric atresia is described in OMIM entry number 612138 (locus and gene 8q24 (PLEC1)).

Epidermolysis bullosa simplex of Ogna is described in OMIM entry number 131950 (locus and gene 8q24 (PLEC1)). Has onset in infancy, presenting with seasonal blistering on acral areas during summer months.

### Junctional Epidermolysis Bullosa

[The text below is adapted from Junctional epidermolysis bullosa (medicine). (2014, January 23). In Wikipedia, The Free Encyclopedia. Retrieved 11:50, Sep. 11, 2014, from http://en.wikipedia.org/w/index.php?title=Junctional_epidermolysis_bullosa_(medicine)&oldid=591972576]

Junctional epidermolysis bullosa is an inherited disease affecting laminin and collagen. This disease is characterised by blister formation within the lamina lucida of the basement membrane zone. and is inherited in an autosomal recessive manner. It also presents with blisters at the site of friction, especially on the hands and feet, and has variants that can occur in children and adults. Less than one per million people are estimated to have this form of epidermolysis bullosa.

Classification of Junctional Epidermolysis Bullosa

Junctional epidermolysis bullosa may be divided into multiple types:

Junctional epidermolysis bullosa with pyloric atresia is described in OMIM entry number 226730 (locus and gene 17q11-qter, 2q31.1 ITGB4, ITGA6).

Junctional epidermolysis bullosa, Herlitz type is described in OMIM entry number 226700 (locus and gene 18q11.2, 1q32, 1q25-q31 LAMA3, LAMB3, LAMC2).

Epidermolysis bullosa, junctional, non-Herlitz types (Generalized atrophic benign epidermolysis bullosa, Mitis junctional epidermolysis bullosa) is described in OMIM entry number 226650 (locus and gene 18q11.2, 1q32, 17q11-qter, 1q25-q31, 10q24.3 LAMA3, LAMB3, LAMC2, COL17A1, ITGB4).

Junctional Epidermolysis Bullosa with Pyloric Atresia

Junctional epidermolysis bullosa with pyloric atresia is a rare autosomal recessive form of junctional epidermolysis bullosa that presents at birth with severe mucocutaneous fragility and gastric outlet obstruction. It can be associated with ITGB4 or ITGA6.

Junctional Epidermolysis Bullosa Gravis (Herlitz Type)

Junctional epidermolysis bullosa gravis (also known as “Herlitz disease”, “Herlitz syndrome,” and “Lethal junctional epidermolysis bullosa”) is the most lethal type of epidermolysis bullosa, a skin condition in which most patients do not survive infancy, characterized by blistering at birth with severe and clinically distinctive perorificial granulation tissue.

Non-Herlitz Type

These include:

Generalized atrophic benign epidermolysis bullosa is a skin condition that is characterized by onset at birth, generalized blisters and atrophy, mucosal involvement, and thickened, dystrophic, or absent nails.

Mitis junctional epidermolysis bullosa (also known as “Nonlethal junctional epidermolysis bullosa”) is a skin condition characterized by scalp and nail lesions, also associated with periorificial nonhealing erosions. Mitis junctional epidermolysis bullosa is most commonly seem in children between the ages of 4 and 10 years old.

Cicatricial junctional epidermolysis bullosa is a skin condition characterized by blisters that heal with scarring. It was characterized in 1985.

Pathophysiology of Junctional Epidermolysis Bullosa

α6β4 integrin is a transmembrane protein found in hemidesmosomes. As a heterodimer molecule containing two polypeptide chains its extracellular domain enters the basal lamina and interacts with type IV collagen suprastructure containing laminins (laminin-5), entactin/nidongen or the perlecan. on the extracellular surface of the hemidesmosome, laminin-5 molecules form threadlike anchoring filaments that extend from the integrin molecules to the structure of the basement membrane of epithelial adhesion. Mutation of the genes encoding laminin-5 chains results in junctional epidermolysis bullosa.

### Dystrophic Epidermolysis Bullosa

[The text below is adapted from Epidermolysis bullosa dystrophica. (2014, January 18). In Wikipedia, The Free Encyclopedia. Retrieved 11:50, Sep. 11, 2014, from http://en.wikipedia.org/w/index.php?title=Epidermolysis_bullosa_dystrophica&oldid=591217345]

Dystrophic epidermolysis bullosa is an inherited variant affecting the skin and other organs. “Butterfly children” is the term given to those born with the disease, as their skin is seen to be as delicate and fragile as a butterfly's wings. Dystrophic epidermolysis bullosa is caused by genetic defects (or mutations) within the human COL7A1 gene encoding the protein type VII collagen (collagen VII). DEB-causing mutations can be either autosomal dominant or autosomal recessive.

Classification of Dystrophic Epidermolysis Bullosa

Dystrophic epidermolysis bullosa may be divided into multiple types:

Dominant dystrophic epidermolysis bullosa (DDEB) is described in OHIM entry number 131750 (locus and gene 3p21.3 (COL7A1)).

Also known as “Cockayne-Touraine disease”, this variant is characterized by vesicles and bullae on the extensor surfaces of the extremities. is described in OHIM entry number.

Recessive dystrophic epidermolysis bullosa (RDEB) is described in OHIM entry number 226600 (locus and gene 11q22-q23 (COL7A1), 3p21.3 (MMP1)).

Also known as “Hallopeau-Siemens variant of epidermolysis bullosa” and “Hallopeau-Siemens disease”, this variant results from mutations in the gene encoding type VII collagen, COL7A1, characterized by debilitating oral lesions that produce pain, scarring, and microstomia. It is named for François Henri Hallopeau and Hermann Werner Siemens. is described in OHIM entry number.

Epidermolysis bullosa dystrophica, pretibial is described in OHIM entry number 131850 (locus and gene 3p21.3 (COL7A1)).

Epidermolysis bullosa pruriginosa is described in OHIM entry number 604129 (locus and gene 3p21.3 (COL7A1)).

Epidermolysis bullosa with congenital localized absence of skin and deformity of nails is described in OHIM entry number 132000 (locus and gene 3p21.3 (COL7A1)).

Transient bullous dermolysis of the newborn (TBDN) is described in OHIM entry number 131705 (locus and gene 3p21.3 (COL7A1)).

Causes of Dystrophic Epidermolysis Bullosa

DEB is caused by genetic defects (or mutations) within the human COL7A1 gene encoding the protein type VII collagen (collagen VII). DEB-causing mutations can be either dominant or recessive.

Most families with family members with this condition have distinct mutations.

Collagen VII is a very large molecule (300 kDa) that dimerizes to form a semicircular looping structure: the anchoring fibril. Anchoring fibrils are thought to form a structural link between the epidermal basement membrane and the fibrillar collagens in the upper dermis.

Signs and Symptoms of Dystrophic Epidermolysis Bullosa

The deficiency in anchoring fibrils impairs the adherence between the epidermis and the underlying dermis. The skin of DEB patients is thus highly susceptible to severe blistering.

Collagen VII is also associated with the epithelium of the esophageal lining, and DEB patients may suffer from chronic scarring, webbing, and obstruction of the esophagus. Affected individuals are often severely malnourished due to trauma to the oral and esophageal mucosa and require feeding tubes for nutrition. They also suffer from iron-deficiency anemia of uncertain origin, which leads to chronic fatigue.

Open wounds on the skin heal slowly or not at all, often scarring extensively, and are particularly susceptible to infection. Many individuals bathe in a bleach and water mixture to fight off these infections.

The chronic inflammation leads to errors in the DNA of the affected skin cells, which in turn causes squamous cell carcinoma (SCC). The majority of these patients die before the age of 30, either of SCC or complications related to DEB.

Pathophysiology of Dystrophic Epidermolysis Bullosa

In the absence of mutations of the COL7A1 gene, an autoimmune response against type VII collagen can result in an acquired form of epidermolysis bullosa called epidermolysis bullosa acquisita.

There exist other types of inherited epidermolysis bullosa, junctional epidermolysis bullosa and epidermolysis bullosa simplex, which are not related to type VII collagen deficiency. These arise from mutations in the genes encoding other proteins of the epidermis or the basement membrane at the junction between the epidermis and the dermis.

### Exosomes

Exosomes are small membrane vesicles formed in late endocytic compartments (multivesicular bodies) first described to be secreted by reticulocytes in 1983 and subsequently found to be secreted by many cells types including various haematopoietic cells, tumours of haematopoietic or non-haematopoietic origin and epithelial cells. They are distinct entities from the more recently described ‘ribonuclease complex’ also named exosome.

Exosomes may be defined by a number of morphological and biochemical parameters. Accordingly, the exosome described here may comprise one or more of these morphological or biochemical parameters.

Exosomes are classically defined as “saucer-like” vesicles or a flattened sphere limited by a lipid bilayer with diameters of 40-100 nm and are formed by inward budding of the endosomal membrane. Like all lipid vesicles and unlike protein aggregates or nucleosomal fragments that are released by apoptotic cells, exosomes have a density of ˜1.13-1.19 g/ml and float on sucrose gradients. Exosomes are enriched in cholesterol and sphingomyelin, and lipid raft markers such as GM1, GM3, flotillin and the src protein kinase Lyn suggesting that their membranes are enriched in lipid rafts.

The molecular composition of exosomes from different cell types and of different species has been examined. In general, exosomes contain ubiquitous proteins that appear to be common to all exosomes and proteins that are cell-type specific. Also, proteins in exosomes from the same cell-type but of different species are highly conserved. The ubiquitous exosome-associated proteins include cytosolic proteins found in cytoskeleton e.g. tubulin, actin and actin-binding proteins, intracellular membrane fusions and transport e.g. annexins and rab proteins, signal transduction proteins e.g. protein kinases, 14-3-3 and heterotrimeric G proteins, metabolic enzymes e.g. peroxidases, pyruvate and lipid kinases, and enolase-1 and the family of tetraspanins e.g. CD9, CD63, CD81 and CD82. The tetraspannins are highly enriched in exosomes and are known to be involved in the organization of large molecular complexes and membrane subdomains.

Examples of cell-type specific proteins in exosomes are MEW class II molecules in exosomes from MEW class II-expressing cells, CD86 in dendritic cell-derived exosomes, T-cell receptors on T-cell-derived exosomes etc. Notably, exosomes do not contain proteins of nuclear, mitochondrial, endoplasmic-reticulum or Golgi-apparatus origin. Also, highly abundant plasma membrane proteins are absent in exosomes suggesting that they are not simply fragments of the plasma membrane. Many of the reported ubiquitous exosome-associated proteins are also present in the proteomic profile of the hESC-MSC secretion.

Exosomes are also known to contain mRNA and microRNA, which can be delivered to another cell, and can be functional in this new location. The physiological functions of exosome remain poorly defined. It is thought to help eradicate obsolete proteins, recycle proteins, mediate transmission of infectious particles such as prions and viruses, induce complement resistance, facilitate immune cell-cell communication and transmit cell signaling. Exosomes have been used in immunotherapy for treatment of cancer.

### Compositions for Promoting, Restoring or Enhancing Homeostasis

We provide compositions for promoting, restoring or enhancing homeostasis. Such compositions may comprise exosomes, such as exosomes derived from mesenchymal stem cells.

They may comprise mesenchymal stem cell conditioned medium (or even mesenchymal stem cells themselves).

For example, the exosomes, conditioned media and/or mesenchymal stem cells may be mammalian exosomes, conditioned media and/or mesenchymal stem cells, such as human exosomes, conditioned media and/or mesenchymal stem cells.

The exosome composition may be made from an exosome. The exosome may be derivable from a stem cell such as a mesenchymal stem cell (MSC), as described below and in WO 2009/105044.

The exosome may be derivable from a mesenchymal stem cell by any of several means, for example by secretion, budding or dispersal from the mesenchymal stem cell. For example, the exosome may be produced, exuded, emitted or shed from the mesenchymal stem cell. Where the mesenchymal stem cell is in cell culture, the exosome may be secreted into the cell culture medium.

The exosome may in particular comprise a vesicle.

The exosome may comprise vesicles or a flattened sphere limited by a lipid bilayer. The exosome may comprise diameters of 40-100 nm. The exosome may be formed by inward budding of the endosomal membrane. The exosome may have a density of ˜1.13-1.19 g/ml and may float on sucrose gradients. The exosome may be enriched in cholesterol and sphingomyelin, and lipid raft markers such as GM1, GM3, flotillin and the src protein kinase Lyn. The exosome may comprise one or more proteins present in mesenchymal stem cells or mesenchymal stem cell conditioned medium (MSC-CM), such as a protein characteristic or specific to the MSC or MSC-CM. They may comprise RNA, for example miRNA.

We provide a exosome which comprises one or more genes or gene products found in mesenchymal stem cells or medium which is conditioned by culture of mesenchymal stem cells, for use in promoting, restoring or enhancing homeostasis. The exosome may comprise molecules secreted by the mesenchymal stem cell. Such an exosome, and combinations of any of the molecules comprised therein, including in particular proteins or polypeptides, may be used for any of the methods described in this document.

The exosome may comprise a cytosolic protein found in cytoskeleton e.g. tubulin, actin and actin-binding proteins, intracellular membrane fusions and transport e.g. annexins and rab proteins, signal transduction proteins e.g. protein kinases, 14-3-3 and heterotrimeric G proteins, metabolic enzymes e.g. peroxidases, pyruvate and lipid kinases, and enolase-1 and the family of tetraspanins e.g. CD9, CD63, CD81 and CD82. In particular, the exosome may comprise one or more tetraspanins. The exosomes may comprise mRNA and/or microRNA.

The exosome may be something that is isolatable from a mesenchymal stem cell (MSC) or mesenchymal stem cell conditioned medium (MSC-CM). The exosome may be responsible for at least an activity of the MSC or MSC-CM. The exosome may be responsible for, and carry out, substantially most or all of the functions of the MSC or MSC-CM. For example, the exosome may be a substitute (or biological substitute) for the MSC or MSC-CM.

The exosome preferably has at least one property of a mesenchymal stem cell. The exosome may have a biological property, such as a biological activity. The exosome may have any of the biological activities of an mesenchymal stem cell. The exosome may for example have a therapeutic or restorative activity of an mesenchymal stem cell, such as activity in promoting, restoring or enhancing homeostasis.

The exosome may be isolated from a mesenchymal stem cell conditioned medium (MSC-CM).

Mesenchymal Stem Cell Conditioned Medium (MSC-CM)

The conditioned cell culture medium such as a Mesenchymal Stem Cell Conditioned Medium (MSC-CM) may be obtained by culturing a mesenchymal stem cell (MSC), a descendent thereof or a cell line derived therefrom in a cell culture medium; and isolating the cell culture medium. The mesenchymal stem cell may be produced by a process comprising obtaining a cell by dispersing a embryonic stem (ES) cell colony. The cell, or a descendent thereof, may be propagated in the absence of co-culture in a serum free medium comprising FGF2.

Mesenchymal Stem Cell Exosome

Mesenchymal stem cell exosomes are described in detail in International Patent Publication Number WO 2009/105044.

The exosome may be produced or isolated in a number of ways. Such a method may comprise isolating the exosome from a mesenchymal stem cell (MSC). Such a method may comprise isolating the exosome from an mesenchymal stem cell conditioned medium (MSC-CM).

The exosome may be isolated for example by being separated from non-associated components based on any property of the exosome. For example, the exosome may be isolated based on molecular weight, size, shape, composition or biological activity.

The conditioned medium may be filtered or concentrated or both during, prior to or subsequent to separation. For example, it may be filtered through a membrane, for example one with a size or molecular weight cut-off. It may be subject to tangential force filtration or ultrafiltration.

For example, filtration with a membrane of a suitable molecular weight or size cutoff, as described in the Assays for Molecular Weight elsewhere in this document, may be used.

The conditioned medium, optionally filtered or concentrated or both, may be subject to further separation means, such as column chromatography. For example, high performance liquid chromatography (HPLC) with various columns may be used. The columns may be size exclusion columns or binding columns.

One or more properties or biological activities of the exosome may be used to track its activity during fractionation of the mesenchymal stem cell conditioned medium (MSC-CM). As an example, light scattering, refractive index, dynamic light scattering or UV-visible detectors may be used to follow the exosome. For example, a therapeutic activity such as cardioprotective activity may be used to track the activity during fractionation.

The following paragraphs provide a specific example of how a mesenchymal stem cell exosome may be obtained.

A mesenchymal stem cell exosome may be produced by culturing mesenchymal stem cells in a medium to condition it. The mesenchymal stem cells may comprise HuES9.E1 cells. The medium may comprise DMEM. The DMEM may be such that it does not comprise phenol red. The medium may be supplemented with insulin, transferrin, or selenoprotein (ITS), or any combination thereof. It may comprise FGF2. It may comprise PDGF AB. The concentration of FGF2 may be about 5 ng/ml FGF2. The concentration of PDGF AB may be about 5 ng/ml. The medium may comprise glutamine-penicillin-streptomycin or □-mercaptoethanol, or any combination thereof.

The cells may be cultured for about 1, 2, 3, 4, 5, 6, 7, 8, 9, 10 days or more, for example 3 days. The conditioned medium may be obtained by separating the cells from the medium. The conditioned medium may be centrifuged, for example at 500 g. it may be concentrated by filtration through a membrane. The membrane may comprise a >1000 kDa membrane. The conditioned medium may be concentrated about 50 times or more.

The conditioned medium may be subject to liquid chromatography such as HPLC. The conditioned medium may be separated by size exclusion. Any size exclusion matrix such as Sepharose may be used. As an example, a TSK Guard column SWXL, 6×40 mm or a TSK gel G4000 SWXL, 7.8×300 mm may be employed. The eluent buffer may comprise any physiological medium such as saline. It may comprise 20 mM phosphate buffer with 150 mM of NaCl at pH 7.2. The chromatography system may be equilibrated at a flow rate of 0.5 ml/min. The elution mode may be isocratic. UV absorbance at 220 nm may be used to track the progress of elution. Fractions may be examined for dynamic light scattering (DLS) using a quasi-elastic light scattering (QELS) detector.

Fractions which are found to exhibit dynamic light scattering may be retained. For example, a fraction which is produced by the general method as described above, and which elutes with a retention time of 11-13 minutes, such as 12 minutes, is found to exhibit dynamic light scattering. The rₕ of exosomes in this peak is about 45-55 nm. Such fractions comprise mesenchymal stem cell exosomes.

Exosome Molecular Weight

The exosome may have a molecular weight of greater than 100 kDa. It may have a molecular weight of greater than 500 kDa. For example, it may have a molecular weight of greater than 1000 kDa.

The molecular weight may be determined by various means. In principle, the molecular weight may be determined by size fractionation and filtration through a membrane with the relevant molecular weight cut-off. The exosome size may then be determined by tracking segregation of component proteins with SDS-PAGE or by a biological assay.

Assay of Molecular Weight by SDS-PAGE

The exosome may have a molecular weight of greater than 100 kDa. For example, the exosome may be such that most proteins of the exosome with less than 100 kDa molecular weight segregate into the greater than 100 kDa molecular weight retentate fraction, when subject to filtration. Similarly, when subjected to filtration with a membrane with a 500 kDa cut off, most proteins of the exosome with less than 500 kDa molecular weight may segregate into the greater than 500 kDa molecular weight retentate fraction. This indicates that the exosome may have a molecular weight of more than 500 kDa.

Assay of Molecular Weight by Biological Activity

The exosome may have a molecular weight of more than 1000 kDa. For example, the exosome may be such that when subject to filtration with a membrane with a molecular weight cutoff of 1000 kDa, the relevant biological activity substantially or predominantly remains in the retentate fraction. Alternatively or in addition, biological activity may be absent in the filtrate fraction. The biological activity may comprise any of the biological activities of the exosome described elsewhere in this document.

Assay of Molecular Weight by Infarct Size

For example, the biological activity may comprise reduction of infarct size, as assayed in any suitable model of myocardia ischemia and reperfusion injury. For example, the biological activity may be assayed in a mouse or pig model, as described in WO 2009/105044.

In summary, myocardial ischemia is induced by 30 minutes left coronary artery (LCA) occlusion by suture ligation and reperfusion is initiated by removal of suture. Mice are treated with liquid containing the exosomes (such as unfractionated MSC-CM), filtrate (such as <100 or 1,000 kD fraction), retentate (such as >1000 kD retentate) or saline intravenously via the tail vein, 5 minutes before reperfusion. 24 hours later, the hearts are excised. Before excision, the Area At Risk (AAR) is determined by religating the LCA and then perfusing Evans blue through the aorta.

AAR is defined as the area not stained by the dye and is expressed as a percentage of the left ventricular wall area. Infarct size is assessed 24 hours later using Evans blue and TTC. Where the relative infarct size is significantly reduced in animals treated with mesenchymal stem cell conditioned medium (MSC-CM) and the retentate (such as a >1000 kD) fraction when compared to saline, this indicates that the exosome has a molecular weight which is higher than the relevant cutoff of the membrane (e.g., greater than 1000 kDa).

Exosome Size

The exosome may have a size of greater than 2 nm. The exosome may have a size of greater than 5 nm, 10 nm, 20 nm, 30 nm, 40 nm or 50 nm. The exosome may have a size of greater than 100 nm, such as greater than 150 nm. The exosome may have a size of substantially 200 nm or greater.

The exosome may have a range of sizes, such as between 2 nm to 20 nm, 2 nm to 50 nm, 2 nm to 100 nm, 2 nm to 150 nm or 2 nm to 200 nm. The exosome may have a size between 20 nm to 50 nm, 20 nm to 100 nm, 20 nm to 150 nm or 20 nm to 200 nm. The exosome may have a size between 50 nm to 100ηm, 50ηm to 150 nm or 50 nm to 200 nm. The exosome may have a size between 100 nm to 150 nm or 100 nm to 200 nm. The exosome may have a size between 150 nm to 200 nm.

The size may be determined by various means. In principle, the size may be determined by size fractionation and filtration through a membrane with the relevant size cut-off. The exosome size may then be determined by tracking segregation of component proteins with SDS-PAGE or by a biological assay.

The size may also be determined by electron microscopy.

The size may comprise a hydrodynamic radius. The hydrodynamic radius of the exosome may be below 100 nm. It may be between about 30 nm and about 70 nm. The hydrodynamic radius may be between about 40 nm and about 60 nm, such as between about 45 nm and about 55 nm. The hydrodynamic radius may be about 50 nm.

The hydrodynamic radius of the exosome may be determined by any suitable means, for example, laser diffraction or dynamic light scattering. An example of a dynamic light scattering method to determine hydrodynamic radius is described in WO 2009/105044.

### Obtaining Exosomes

Exosomes may be obtained by any of various means described in the art. Exosomes may for example be obtained from mesenchymal stem cells, such as from medium conditioned by mesenchymal stem cells.

Obtaining Exosomes from Mesenchymal Stem Cell (MSC) Conditioned Medium

Mesenchymal stem cell particles such as exosomes may be isolated or produced, using the methods described here, from mesenchymal stem cell conditioned medium (MSC-CM).

MSCs suitable for use in the production of conditioned media and exosomes may be made by any method known in the art.

In particular, MSCs may be made by propagating a cell obtained by dispersing a embryonic stem (ES) cell colony, or a descendent thereof, in the absence of co-culture in a serum free medium comprising FGF2. This is described in detail in the sections below.

Methods of obtaining mesenchymal stem cells (MSC) or MSC-like cells from hESCs may involve either transfection of a human telomerase reverse transcriptase (hTERT) gene into differentiating hESCs (Xu et al., 2004) or coculture with mouse OP9 cell line (Barberi et al., 2005). The use of exogenous genetic material and mouse cells in these derivation protocols introduces unacceptable risks of tumorigenicity or infection of xenozootic infectious agents.

The exosomes may therefore be made from MSCs derived by the use of a clinically relevant and reproducible protocol for isolating similar or identical (such as homogenous) MSC populations from differentiating hESCs. In general, the method comprises dispersing a embryonic stem (ES) cell colony into cells. The cells are then plated out and propagated. The cells are propagated in the absence of co-culture in a serum free medium comprising fibroblast growth factor 2 (FGF2), in order to obtain mesenchymal stem cells (MSCs).

Thus, the protocol does not require serum, use of mouse cells or genetic manipulations and requires less manipulations and time, and is therefore highly scalable. The protocol may be used for the isolation of MSCs from two different hESC lines, HuES9 and H-1 and also a third one, Hes-3. Human ES cell derived MSCs (hESC-MSCs) obtained by the methods and compositions described here are remarkably similar to bone-marrow derived MSCs (BM-MSCs).

The embryonic stem cell culture may comprise a human embryonic stem cell (hESC) culture.

In a one embodiment, a method of generating mesenchymal stem cells (MSC) comprises trypsinizing and propagating hESCs without feeder support in media supplemented with FGF2 and optionally PDGF AB before sorting for CD105+CD24− cells.

The method may comprise sorting for CD105+, CD24− cells from trypsinized hESCs one week after feeder-free propagation in a media supplemented with FGF2 and optionally PDGF AB will generate to generate a hESC-MSC cell culture in which at least some, such as substantially all, or all cells are similar or identical (such as homogenous) to each other.

The MSCs produced by this method may be used to produce mesenchymal stem cell conditioned medium (MSC-CM), from which the exosomes may be isolated.

Disaggregating Embryonic Stem Cell Colonies

One method of producing mesenchymal stem cells may comprise dispersing or disaggregating an embryonic stem cell colony into cells.

The embryonic stem cell colony may comprise a huES9 colony (Cowan C A, Klimanskaya I, McMahon J, Atienza J, Witmyer J, et al. (2004) Derivation of embryonic stem-cell lines from human blastocysts. N Engl J Med 350: 1353-1356) or a H1 ESC colony (Thomson J A, Itskovitz-Eldor J, Shapiro S S, Waknitz M A, Swiergiel J J, et al. (1998) Embryonic Stem Cell Lines Derived from Human Blastocysts. Science 282: 1145-1147.).

The cells in the colony may be disaggregated or dispersed to a substantial extent, i.e., at least into clumps. The colony may be disaggregated or dispersed to the extent that all the cells in the colony are single, i.e., the colony is completely disaggregated.

The disaggregation may be achieved with a dispersing agent.

The dispersing agent may be anything that is capable of detaching at least some embryonic stem cells in a colony from each other. The dispersing agent may comprise a reagent which disrupts the adhesion between cells in a colony, or between cells and a substrate, or both. The dispersing agent may comprise a protease.

The dispersing agent may comprise trypsin. The treatment with trypsin may last for example for 3 minutes or thereabouts at 37 degrees C. The cells may then be neutralised, centrifuged and resuspended in medium before plating out.

The method may comprise dispersing a confluent plate of human embryonic stem cells with trypsin and plating the cells out.

The disaggregation may comprise at least some of the following sequence of steps: aspiration, rinsing, trypsinization, incubation, dislodging, quenching, re-seeding and aliquoting. The following protocol is adapted from the Hedrick Lab, UC San Diego (http://hedricklab.ucsd.edu/Protocol/COSCell.html).

In the aspiration step, the media is aspirated or generally removed from the vessel, such as a flask. In the rinsing step, the cells are rinsed with a volume, for example 5-10 mls, of a buffered medium, which is may be free from Ca²⁺ and Mg²⁺. For example, the cells may be rinsed with calcium and magnesium free PBS. In the trypsinization step, an amount of dispersing agent in buffer is added to the vessel, and the vessel rolled to coat the growing surface with the dispersing agent solution. For example, 1 ml of trypsin in Hank's BSS may be added to a flask.

In the incubation step, the cells are left for some time at a maintained temperature. For example, the cells may be left at 37° C. for a few minutes (e.g., 2 to 5 minutes). In the dislodging step, the cells may be dislodged by mechanical action, for example by scraping or by whacking the side of the vessel with a hand. The cells should come off in sheets and slide down the surface.

In the quenching step, a volume of medium is added to the flask. The medium may comprise a neutralising agent to stop the action of the dispersing agent. For example, if the dispersing agent is a protease such as trypsin, the medium may contain a protein, such as a serum protein, which will mop up the activity of the protease. In a particular example, 3 ml of serum containing cell culture medium is added to the flask to make up a total of 4 mls. The cells may be pipetted to dislodge or disperse the cells.

In the re-seeding step, the cells are re-seeded into fresh culture vessels and fresh medium added. A number of re-seedings may be made at different split ratios. For example, the cells may be reseeded at 1/15 dilution and 1/5 dilution. In a particular example, the cells may be re-seeded by adding 1 drop of cells into a 25 cm² flask and 3 drops into another to re-seed the culture, and 7-8 mls media is then added to each to provide for 1/15 dilution and 1/5 dilution from for example a 75 cm² flask. In the aliquoting step, the cells may be aliquoted into new dishes or whatever split ratio is desired, and media added.

In a specific embodiment, the method includes the following steps: human ES cells are first grown suspended in non-adherent manner to form embryoid bodies (EBs). 5-10 day old EBs are then trypsinized before plating as adherent cells on gelatine coated tissue culture plates.

Maintenance as Cell Culture

The disaggregated cells may be plated and maintained as a cell culture.

The cells may be plated onto a culture vessel or substrate such as a gelatinized plate. Crucially, the cells are grown and propagated without the presence of co-culture, e.g., in the absence of feeder cells.

The cells in the cell culture may be grown in a serum-free medium which is supplemented by one or more growth factors such as fibroblast growth factor 2 (FGF2) and optionally platelet-derived growth factor AB (PDGF AB), at for example 5 ng/ml. The cells in the cell culture may be split or subcultured 1:4 when confluent, by treatment with trypsin, washing and replating.

Absence of Co-Culture

The cells may be cultured in the absence of co-culture. The term “co-culture” refers to a mixture of two or more different kinds of cells that are grown together, for example, stromal feeder cells.

Thus, in typical ES cell culture, the inner surface of the culture dish is usually coated with a feeder layer of mouse embryonic skin cells that have been treated so they will not divide. The feeder layer provides an adherent surface to enable the ES cells to attach and grow. In addition, the feeder cells release nutrients into the culture medium which are required for ES cell growth. In the methods and compositions described here, the ES and MSC cells may be cultured in the absence of such co-culture.

The cells may be cultured as a monolayer or in the absence of feeder cells. The embryonic stem cells may be cultured in the absence of feeder cells to establish mesenchymal stem cells (MSC).

The dissociated or disaggregated embryonic stem cells may be plated directly onto a culture substrate. The culture substrate may comprise a tissue culture vessel, such as a Petri dish. The vessel may be pre-treated. The cells may be plated onto, and grow on, a gelatinised tissue culture plate.

An example protocol for the gelatin coating of dishes follows. A solution of 0.1% gelatin in distilled water is made and autoclaved. This may be stored at room temp. The bottom of a tissue culture dish is covered with the gelatin solution and incubated for 5-15 min. Remove gelatin and plates are ready to use. Medium should be added before adding cells to prevent hypotonic lysis.

Serum Free Media

The dissociated or disaggregated embryonic stem cells may be cultured in a medium which may comprise a serum-free medium.

The term “serum-free media” may comprise cell culture media which is free of serum proteins, e.g., fetal calf serum. Serum-free media are known in the art, and are described for example in U.S. Pat. Nos. 5,631,159 and 5,661,034. Serum-free media are commercially available from, for example, Gibco-BRL (Invitrogen).

The serum-free media may be protein free, in that it may lack proteins, hydrolysates, and components of unknown composition. The serum-free media may comprise chemically-defined media in which all components have a known chemical structure. Chemically-defined serum-free media is advantageous as it provides a completely defined system which eliminates variability allows for improved reproducibility and more consistent performance, and decreases possibility of contamination by adventitious agents.

The serum-free media may comprise Knockout DMEM media (Invitrogen-Gibco, Grand Island, N.Y.).

The serum-free media may be supplemented with one or more components, such as serum replacement media, at a concentration of for example, 5%, 10%, 15%, etc. The serum-free media may comprise or be supplemented with 10% serum replacement media from Invitrogen-Gibco (Grand Island, N.Y.).

Growth Factor

The serum-free medium in which the dissociated or disaggregated embryonic stem cells are cultured may comprise one or more growth factors. A number of growth factors are known in the art, including PDGF, EGF, TGF-a, FGF, NGF, Erythropoietin, TGF-b, IGF-I and IGF-II.

The growth factor may comprise fibroblast growth factor 2 (FGF2). The medium may also contain other growth factors such as platelet-derived growth factor AB (PDGF AB). Both of these growth factors are known in the art. The method may comprise culturing cells in a medium comprising both FGF2 and PDGF AB.

Alternatively, or in addition, the medium may comprise or further comprise epidermal growth factor (EGF). Use of EGF may enhance growth of MSCs. EGF may be used at any suitable concentration, for example 5-10 ng/ml EGF. EGF may be used in place of PDGF. EGF is a protein well known in the art, and is referred to as symbol EGF, Alt. Symbols URG, Entrez 1950, HUGO 3229, OMIM 131530, RefSeq NM_001963, UniProt P01133.

Thus, we disclose the use of media comprising (i) FGF2, (ii) FGF2 and PDGF and (iii) FGF2 and EGF and other combinations.

FGF2 is a wide-spectrum mitogenic, angiogenic, and neurotrophic factor that is expressed at low levels in many tissues and cell types and reaches high concentrations in brain and pituitary. FGF2 has been implicated in a multitude of physiologic and pathologic processes, including limb development, angiogenesis, wound healing, and tumor growth. FGF2 may be obtained commercially, for example from Invitrogen-Gibco (Grand Island, N.Y.).

Platelet Derived Growth Factor (PDGF) is a potent mitogen for a wide range of cell types including fibroblasts, smooth muscle and connective tissue. PDGF, which is composed of a dimer of two chains termed the A chain and B chain, can be present as AA or BB homodimers or as an AB heterodimer. Human PDGF-AB is a 25.5 kDa homodimer protein consisting of 13.3 kDa A chain and 12.2 B chain. PDGF AB may be obtained commercially, for example from Peprotech (Rocky Hill, N.J.).

The growth factor(s), such as FGF2 and optionally PDGF AB, may be present in the medium at concentrations of about 100 pg/ml, such as about 500 pg/ml, such as about 1 ng/ml, such as about 2 ng/ml, such as about 3 ng/ml, such as about 4 ng/ml, such as about 5 ng/ml. In some embodiments, the medium contains FGF2 at about 5 ng/ml. The medium may also contain PDGF AB, such as at about 5 ng/ml.

Splitting Cells

Cells in culture will generally continue growing until confluence, when contact inhibition causes cessation of cell division and growth. Such cells may then be dissociated from the substrate or flask, and “split”, subcultured or passaged, by dilution into tissue culture medium and replating.

The methods and compositions described here may therefore comprise passaging, or splitting during culture. The cells in the cell culture may be split at a ratio of 1:2 or more, such as 1:3, such as 1:4, 1:5 or more. The term “passage” designates the process consisting in taking an aliquot of a confluent culture of a cell line, in inoculating into fresh medium, and in culturing the line until confluence or saturation is obtained.

Selection, Screening or Sorting Step

The method may further comprise a selection or sorting step, to further isolate or select for mesenchymal stem cells.

The selection or sorting step may comprise selecting mesenchymal stem cells (MSC) from the cell culture by means of one or more surface antigen markers. The use of a selection or sorting step further enhances the stringency of sorting and selection specificity for MSCs and furthermore potentially reduces possible contamination from embryonic stem cells such as hESCs and other hESC-derivatives from the starting material. This would then further reduce the risk of teratoma formation and further increase the clinical relevance of the protocol we describe.

A number of methods are known for selection or sorting based on antigen expression, and any of these may be used in the selection or sorting step described here. The selection or sorting may be achieved by means of fluorescence activated cell sorting (FACS). Thus, as known in the art, FACS involves exposing cells to a reporter, such as a labelled antibody, which binds to and labels antigens expressed by the cell. Methods of production of antibodies and labelling thereof to form reporters are known in the art, and described for example in Harlow and Lane. The cells are then passed through a FACS machine, which sorts the cells from each other based on the labelling. Alternatively or in addition, magnetic cell sorting (MACS) may be employed to sort the cells.

We have realised that while a number of candidate surface antigens known to be associated with MSCs e.g. CD105, CD73, ANPEP, ITGA4 (CD49d), PDGFRA, some of the MSC associated surface antigens e.g. CD29 and CD49e are also highly expressed in ES cells such as hESCs and their expression are verified by FACS analysis. The association of a surface antigen with MSCs may not be sufficient to qualify the antigen as a selectable marker for isolating MSCs from ES cells such as hESC. Accordingly, the selection or sorting step may employ antigens which are differentially expressed between MSCs and ES cells.

The selection or sorting step of our method may positively select for mesenchymal stem cells based on the expression of antigens. Such antigens may be identified by, for example, comparing the gene expression profiles of hESCs and hESCMSCs.

The selection or sorting step of our method may positively select for mesenchymal stem cells based on the expression of antigens which are identified as expressed on MSCs, but not expressed on ES cells such as hESCs.

CD73 is highly expressed on MSCs, while being not highly expressed on hESCs. Both CD73 and CD105 are highly expressed surface antigens in MSCs and are among the top 20 highly expressed surface antigens in hESC-MSCs relative to hESC, the use of either CD73 or CD105 (or both) as selectable marker for putative MSCs will be equally effective in sorting for putative MSCs generated by differentiating hESCs.

Alternatively, or in addition, the selection or sorting step may negatively select against antigens based on surface antigens that are highly expressed as surface antigen on embryonic stem cells (ES cells) such as hESCs, and not mesenchymal stem cells e.g., hESC-MSC. Selection or sorting may be based on known or previously identified hESC-specific surface antigens such as MIBP, ITGB1BP3 and PODXL, and CD24.

FACS analysis confirms the expression of CD24 on hESC but not hESC-MSCs. Therefore, CD24 may be used as a negative selection or sorting marker either on its own, or in conjunction with CD105 as a positive selectable marker for isolating putative MSCs from differentiating hESC cultures.

### Mesenchymal Stem Cell Conditioned Media

Methods of production of media conditioned by mesenchymal stem cells are known in the art, and are described in for example International Patent Publication Number WO2008/020815.

Conditioned medium may be made by culturing mesenchymal stem cells in a medium, such as a cell culture medium, for a predetermined length of time. The mesenchymal stem cells may in particular comprise those produced by any of the methods described in this document, or by methods known in the art.

The conditioned medium may be used in therapy as is, or after one or more treatment steps. For example, the conditioned medium may be UV treated, filter sterilised, etc. One or more purification steps may be employed. Exosomes may be obtained from the conditioned medium for use in therapy.

In particular, the conditioned media may be concentrated, for example by dialysis or ultrafiltration. For example, the medium may be concentrated using membrane ultrafiltration with a nominal molecular weight limit (NMWL) of for example 3K.

### Purification of Exosomes

Exosomes may be purified from mesenchymal stem cells by various methods known in the art, for example in International Patent Publication Numbers WO 2009/105044 (size exclusion chromatography) and WO 2012/087241 (ion exchange chromatography).

### Delivery of Exosomes

The exosomes as described in this document may be delivered to the human or animal body by any suitable means.

We therefore describe a delivery system for delivering a particles such as exosomes described in this document to a target cell, tissue, organ, animal body or human body, and methods for using the delivery system to deliver particles to a target, for promoting, restoring or enhancing homeostasis in a biological environment.

The delivery system may comprise a source of particles such as exosomes such as a container containing the particles. The delivery system may comprise a dispenser for dispensing the particles to a target.

Accordingly, we provide a delivery system for delivering a particle such as an exosome as described here to a target, for promoting, restoring or enhancing homeostasis in a biological environment, comprising a source of particles as described in this document together with a dispenser operable to deliver the particles to a target.

We further provide for the use of such a delivery system in a method of delivering a particles to a target, for promoting, restoring or enhancing homeostasis in a biological environment.

Delivery systems for delivering fluid into the body are known in the art, and include injection, surgical drips, catheters (including perfusion catheters) such as those described in U.S. Pat. No. 6,139,524, for example, drug delivery catheters such as those described in U.S. Pat. No. 7,122,019.

Delivery to the lungs or nasal passages, including intranasal delivery, may be achieved using for example a nasal spray, puffer, inhaler, etc as known in the art (for example as shown in United States Design Patent D544,957.

Delivery to the kidneys may be achieved using an intra-aortic renal delivery catheter, such as that described in U.S. Pat. No. 7,241,273.

It will be evident that the particular delivery should be configurable to deliver the required amount of particles at the appropriate interval, in order to achieve optimal treatment.

It will also be evident that the delivery method will depend on the particular organ to which the particles is to be delivered, and the skilled person will be able to determine which means to employ accordingly.

The particles may be used for the treatment or prevention of dermatological conditions e.g. epidermolysis bullosa. Long term delivery of particles may be employed using transdermal microinjection needles until the condition is resolved.

### Therapeutic Methods

An exosome may be orally, topically, or parenterally administered to a subject suspected of or having a need for restoration, enhancement or promotion of homeostasis (including having or being suspected of having any relevant disease, as set out elsewhere in this document).

The individual subject suspected of or having a need for restoration, enhancement or promotion of homeostasis may be suffering or suspected to be suffering from graft versus host disease (GVHD) or epidermolysis bullosa (EB).

Accordingly, an exosome may be administered to a subject suspected of or having graft versus host disease (GVHD) or epidermolysis bullosa (EB) for the treatment or maintenance thereof.

One of skill in the art can determine the therapeutically effective amount of the composition to be administered to a subject based upon several considerations, such as absorption, metabolism, method of delivery, age, weight, disease severity and response to the therapy. Oral administration of the composition includes oral, buccal, enteral or intragastric administration. It is also envisioned that the composition may be used as a food additive. For example, the composition is sprinkled on food or added to a liquid prior to ingestion. Topical administration of the composition includes topical, dermal, epidermal, or subcutaneous administration. Parenteral administration includes, but is not limited to intramuscular, intravenous, intraperitoneal, intraoccular or intraarticular administration or administration into a surgical field.

The exosome may be administered in an effective amount to promote, restore or enhance homeostasis (or to treat or prevent any relevant disease).

Treatment regimens may vary as well, and often depend on the severity of the condition, the health and age of the patient etc. Obviously, certain types of conditions will require more aggressive treatment, while at the same time, certain patients cannot tolerate more taxing protocols. The clinician will be best suited to make such decisions based on the known efficacy and toxicity (if any) of the therapeutic formulations.

The composition may be given in a single dose or multiple doses. The single dose may be administered daily, or multiple times a day, or multiple times a week, or monthly or multiple times a month. The composition may be given in a series of doses. The series of doses may be administered daily, or multiple times a day, weekly, or multiple times a week, or monthly, or multiple times a month. Thus, one of skill in the art realizes that depending upon the condition, health of the subject, etc., the exosome composition described here may be administered for any given period of time until the disease is treated or a symptom is alleviated or homeostasis is enhanced, promoted or restored by at least by 5%, 10%, 20%, 30%, 40%, 50%, 55%, 60%, 65%, 70%, 75%, 80%, 85%, 90%, 95% or 100% or any range in between.

For topical administration, a gel formulation comprising exosomes may be used to coat fibres of an absorbent gauze dressing to form a healing bandage which may then be placed on a wound or skin condition such as epidermolysis bullosa. A low viscosity formulation may be used. The bandage may be prepared by soaking a gauze dressing with an aqueous gel solution comprising exosomes having wound healing activity. The bandage can then be applied to the wound so that the coated fibres of the gauze contacts the wound and stimulate the rate of wound healing.

Where a gel comprising exosomes is applied to an internal or incisional position, the gel forming polymer may be biodegradable. The naturally occurring polymers are generally biodegradable. Examples of these are collagen, the glycosaminoglycans, gelatin and starch. The cellulosics are not biodegradable. The synthetic polymers such as the vinyl polymers are not degradable. The biodegradability of the polymers described herein is well known to those skilled in the art.

We also describe a method of promoting, restoring or enhancing homeostasis (or treatment or prevention of any relevant disease) comprising the step of supplementing the systemic immune system by increasing the amount of exosome in the systemic circulation. The exosomes may be administered via a parenteral route, which includes, but is not limited to intramuscular, intravenous, intraperitoneal, intraoccular, intraarticular or into a surgical field.

We further disclose a method of promoting, restoring or enhancing homeostasis (or treatment or prevention of any relevant disease) comprising the step of supplementing the mucosal immune system by increasing the amount of exosomes in the gastrointestinal tract of the subject.

We describe a method of enhancing the immune system of a subject suffering from a relevant condition by administering to the subject an exosome composition. Depending upon the mode of administration, different arms of the immune system are enhanced. For example, topical administration of the composition results in enhancement of the local immune system. Parenteral administration of the composition results in enhancement of the systemic immune system. Yet further, oral administration of the composition results in enhancement of the mucosal immune system, which can also result in systemic effects as well.

The immune system, whether local, systemic or mucosal, may be enhanced by exosomes stimulating cytokines and/or chemokines. Exemplary cytokines include interleukin-18 and GM-CSF in the gastrointestinal tract, which are known to enhance immune cells or stimulate production of immune cells. For example, interleukin-18 enhances natural killer cells or T lymphocytes. For example, interleukin-18 (IL-18) enhances CD4+, CD8+ and CD3+ cells. It is known by those of skill in the art that IL-18 is a Th.sub.1 cytokine that acts in synergy with interleukin-12 and interleukin-2 in the stimulation of lymphocyte IFN-gamma production. Other cytokines or chemokines may also be enhanced for example, but not limited to IL-12, IL-1b, MIP-3α, MIP-1α, or IFN-gamma. Other cytokines or enzymes may be inhibited for example, but not limited to IL-2, IL-4, IL-5, IL-10, TNF-α, or matrix metalloproteinases. It is further contemplated that IL-18 or GM-CSF stimulate the production or activity of cells involved in repair, for example, but not limited to keratinocytes, endothelial cells, dendritic cells, fibroblasts, and myofibroblasts. Yet further, it is envisioned that exosomes inhibit the production of TNF-alpha, which inhibits cells involved in inflammation.

The local immune system in a subject may be boosted by administering topically a therapeutically effective amount of an exosome composition. Topical administration of an exosome composition may stimulate the production of a cytokine or a chemokine. Exemplary cytokines that can be stimulated by exosomes may include, but are not limited to interleukin-18 (IL-18), interleukin-12 (IL-12), granulocyte/macrophage colony-stimulating factor (GM-CSF), and gamma interferon (IFN-γ). Exemplary chemokines include, but are not limited to macrophage inflammatory protein 3 alpha (MIP-3a), macrophage inflammatory protein 1 alpha (MIP-1a), or macrophage inflammatory protein beta (MIP-10).

The exosome composition may also result in inhibition of a cytokine or chemokine. The cytokines include, but are not limited to interleukin-2 (IL-2), interleukin-4 (IL-4), interleukin-5 (IL-5), interleukin-10 (IL-10), and tumor necrosis factor alpha (TNF-α). Still further, the exosome composition can also inhibit the production of matrix metalloproteinases (MMPs).

Cytokines, for example, interleukin-18 or granulocyte/macrophage colony-stimulating factor, can stimulate the production or activity of immune cells. The immune cells include, but are not limited to T lymphocytes, natural killer cells, macrophages, dendritic cells, and polymorphonuclear cells. More specifically, the polymorphonuclear cells are neutrophils and the T lymphocytes are selected from the group consisting of CD4+, CD8+ and CD3+ T cells.

Cytokines, for example, interleukin-18 or granulocyte/macrophage colony-stimulating factor, can also stimulate the production or activity of cells involved in repair. The cells involved in repair include, but are not limited to keratinocytes, endothelial cells, fibroblasts, dendritic cells, and myofibroblasts. The inhibition of TNF-alpha further inhibits the migration and maturation of dendritic cells. The dendritic cells can be Langerhans cells.

### Administration

We disclose method of promoting, restoring or enhancing homeostasis (or treatment or prevention of any relevant disease), the method comprising the step of administering a therapeutically effective amount of a exosome to a subject to result in an improvement or a remediation of a symptom.

The exosome may be applied in any suitable quantity. For example, a composition containing 10 μg or less, such as 5 μg or less, such as 2 μg or less, such as 1 μg or less, such as 0.5 μg or less, such as 0.3 μg of exosome may be applied to subject.

The pharmaceutical composition may comprise 40 μg/ml or less, 20 μg/ml or less, 8 μg/ml or less, 4 μg/ml or less, 2 μg/ml or less or 1.2 μg/ml or less of exosome.

The composition may be administered for any suitable length of time, such as at least one week to twelve weeks. The amount of exosome that is administered may comprise any suitable amount, such as about 0.0001 milligram to about 100 g per day.

The exosome may be administered to an animal such as a mammal in need thereof. The animal may be a farm animal, such as a goat, horse, pig, or cow; a pet animal, such as a dog or cat; a laboratory animal, such as a mouse, rat, or guinea pig; or a primate, such as a monkey, orangutan, ape, chimpanzee, or human. For example, the mammal may be a human.

The exosome can be incorporated in a pharmaceutical composition suitable for use as a medicament, for human or animal use. Pharmaceutical compositions are described in further detail below.

An effective amount of the exosome, such as in a pharmaceutical composition, may be administered to a human or an animal in need thereof by any of a number of well-known methods. For example, the exosome may be administered systemically or locally, for example by injection.

The systemic administration of the exosome may be by intravenous, subcutaneous, intraperitoneal, intramuscular, intrathecal or oral administration. Alternatively, the exosome may be applied topically in appropriate situations.

An effective amount of a pharmaceutical composition described here may comprise any amount that is effective to achieve its purpose. The effective amount, usually expressed in mg/kg can be determined by routine methods during pre-clinical and clinical trials by those of skill in the art.

Individual

The exosomes are delivered to individuals. As used herein, the term “individual” refers to vertebrates, particularly members of the mammalian species. The term includes but is not limited to domestic animals, sports animals, primates and humans.

### Further Aspects

Further aspects and embodiments of the invention are now set out in the following numbered Paragraphs; it is to be understood that the invention encompasses these aspects:

Paragraph 1. A mesenchymal stem cell for use in a method of promoting, restoring or enhancing homeostasis in a biological environment in need of such, the method comprising exposing the biological environment to the mesenchymal stem cell.

Paragraph 2. A mesenchymal stem cell according to Paragraph 1 for a use specified therein, in which the biological environment comprises an environment in or of a cell, tissue, organ, system or organism.

Paragraph 3. A mesenchymal stem cell according to Paragraph 1 or 2 for a use specified therein, in which the biological environment comprises a cardiovascular system or an immune system of an organism.

Paragraph 4. A mesenchymal stem cell according to Paragraph 1, 2 or 3 for a use specified therein, in which the homeostasis comprises maintenance of a biochemical or a biophysical parameter, or both.

Paragraph 5. A mesenchymal stem cell according to any preceding Paragraph for a use specified therein, in which the homeostasis comprises cell homeostasis, for example maintenance of a parameter selected from the group consisting of: (a) the number of cells; (b) the state of cells; (c) the type of cells; and (d) the composition of an extracellular environment, for example an extracellular matrix.

Paragraph 6. A mesenchymal stem cell according to any preceding Paragraph for a use specified therein, in which the homeostasis comprises maintenance of physiological pH, for example pH 7.4.

Paragraph 7. A mesenchymal stem cell according to any preceding Paragraph for a use specified therein, in which the homeostasis comprises immune homeostasis, for example, maintenance of an immune response.

Paragraph 8. A mesenchymal stem cell according to any preceding Paragraph for a use specified therein, in which the biological environment comprises an individual suffering from: (a) an autoimmune disease, for example Crohn disease or muscular dystrophy; (b) a disease where the pathology includes secondary immune reactivity, for example chronic heart failure, atherosclerosis, a coronary disease or a cerebral artery disease; (c) a neurodegenerative disease; (d) a gerontological condition; or (e) an immune disorder, for example chronic inflammation or reduced immune recognition of self versus non-self.

Paragraph 9. A mesenchymal stem cell for use in a method of promoting, restoring or enhancing homeostasis in an individual suffering from a disease or a tissue injury, the method comprising administering a mesenchymal stem cell to individual.

Paragraph 10. Use of a mesenchymal stem cell in a method of production of adenosine from adenosine monophosphate (AMP).

Paragraph 11. Use of a mesenchymal stem cell in a method of promoting or enhancing phosphorylation or a pro-survival protein kinase, for example Akt, Erk 1 or Erk 2.

Paragraph 12. A method of promoting, restoring or enhancing homeostasis in a biological environment in need of such, the method comprising exposing the biological environment to the mesenchymal stem cell.

Paragraph 13. A method according to Paragraph 12, in which the biological environment comprises an environment in or of a cell, tissue, organ, system, for example a cardiovascular system or an immune system, or an organism.

Paragraph 14. A method according to Paragraph 12 or 13, in which the homeostasis is selected from the group consisting of: (a) maintenance of a biochemical parameter; (b) maintenance of a biophysical parameter; (c) cell homeostasis; (d) maintenance of the number of cells; (e) maintenance of the state of cells; (f) maintenance of the type of cells; (g) maintenance of the composition of an extracellular environment; (h) maintenance of the composition of an extracellular matrix; (i) maintenance of physiological pH, for example pH 7.4; (j) immune homeostasis; and (k) maintenance of an immune response.

Paragraph 15. A method of treatment of an individual suffering from a disease or a tissue injury by promoting, restoring or enhancing homeostasis in the individual, the method comprising administering a mesenchymal stem cell to individual.

Paragraph 16. A method according to Paragraph 16, in which the individual is suffering from a condition selected from the group consisting of: (a) an autoimmune disease, for example Crohn disease or muscular dystrophy; (b) a disease where the pathology includes secondary immune reactivity, for example chronic heart failure, atherosclerosis, a coronary disease or a cerebral artery disease; (c) a neurodegenerative disease; (d) a gerontological condition; or (e) an immune disorder, for example chronic inflammation or reduced immune recognition of self versus non-self.

Paragraph A1. An exosome for use in a method of promoting, restoring or enhancing homeostasis in a biological environment in need of such.

Paragraph A2. An exosome according to Paragraph A1 for a use specified therein, in which the biological environment comprises an environment in or of a cell, tissue, organ, system or organism, and in which the method comprises exposing the biological environment to the exosome.

Paragraph A3. An exosome according to Paragraph A1 or A2 for a use specified therein, in which the homeostasis comprises maintenance of a biochemical or a biophysical parameter, or both.

Paragraph A4. An exosome according to Paragraph A1, A2 or A3 for a use specified therein, in which the homeostasis comprises cardiovascular homeostasis and the biological environment comprises a cardiovascular system of an organism, or in which the homeostasis comprises immune homeostasis and the biological environment comprises an immune system of an organism.

Paragraph A5. An exosome according to any preceding paragraph for a use specified therein, in which the homeostasis comprises cell homeostasis, for example maintenance of a parameter selected from the group consisting of: (a) the number of cells; (b) the state of cells; (c) the type of cells; and (d) the composition of an extracellular environment, for example an extracellular matrix.

Paragraph A6. An exosome according to any preceding paragraph for a use specified therein, in which the homeostasis comprises maintenance of physiological pH, for example pH 7.4.

Paragraph A7. An exosome according to any preceding paragraph for a use specified therein, in which the homeostasis comprises immune homeostasis, for example, maintenance of an immune response.

Paragraph A8. An exosome for use in a method of promoting, restoring or enhancing homeostasis in an individual suffering from a disease or a tissue injury, the method comprising administering an exosome to individual.

Paragraph A9. An exosome according to any preceding paragraph for a use specified therein, in which the biological environment comprises an individual suffering from, or in which the disease comprises: (a) an autoimmune disease, for example Crohn disease or muscular dystrophy; (b) a disease where the pathology includes secondary immune reactivity, for example chronic heart failure, atherosclerosis, a coronary disease or a cerebral artery disease; (c) a neurodegenerative disease; (d) a gerontological condition; (e) an immune disorder, for example chronic inflammation or reduced immune recognition of self versus non-self; (f) Duchenne muscular dystrophy (DMD); (g) graft-versus-host disease (GVHD); or (h) epidermolysis bullosa (EB).

Paragraph A10. An exosome according to any preceding paragraph for a use specified therein, in which the exosome comprises a mesenchymal stem cell exosome.

Paragraph A11. Use of an exosome in a method of production of adenosine from adenosine monophosphate (AMP).

Paragraph A12. Use of an exosome in a method of promoting or enhancing phosphorylation or a pro-survival protein kinase, for example Akt, Erk 1 or Erk 2.

Paragraph A13. A method of promoting, restoring or enhancing homeostasis in a biological environment in need of such, the method comprising exposing the biological environment to an exosome.

Paragraph A14. A method according to Paragraph A13, in which the biological environment comprises an environment in or of a cell, tissue, organ, system, for example a cardiovascular system or an immune system, or an organism.

Paragraph A15. A method according to Paragraph A13 or A14, in which the homeostasis is selected from the group consisting of: (a) maintenance of a biochemical parameter; (b) maintenance of a biophysical parameter; (c) cell homeostasis; (d) maintenance of the number of cells; (e) maintenance of the state of cells; (f) maintenance of the type of cells; (g) maintenance of the composition of an extracellular environment; (h) maintenance of the composition of an extracellular matrix; (i) maintenance of physiological pH, for example pH 7.4; (j) immune homeostasis; and (k) maintenance of an immune response.

Paragraph A16. A method of treatment of an individual suffering from a disease or a tissue injury by promoting, restoring or enhancing homeostasis in the individual, the method comprising administering an exosome to the individual.

Paragraph A17. A method according to Paragraph A16, in which the individual is suffering from a condition selected from the group consisting of: (a) an autoimmune disease, for example Crohn disease or muscular dystrophy; (b) a disease where the pathology includes secondary immune reactivity, for example chronic heart failure, atherosclerosis, a coronary disease or a cerebral artery disease; (c) a neurodegenerative disease; (d) a gerontological condition; or (e) an immune disorder, for example chronic inflammation or reduced immune recognition of self versus non-self.

### EXAMPLES

### Example 1. MSC Exosomes Through CD73-Mediate Hydrolysis of AMP Induced ERK and AKT Pro-Survival Signaling by Adenosine—Increasing AMP Concentration

H9C2 cardiomyocytes (ATCC) were plated onto a 6 well plate at 200,000 cells per well in high-glucose Dulbecco's modified Eagle medium (DMEM) medium containing 10% fetal calf serum, 1% glutamine-penicillin-streptomycin and 1% sodium pyruvate (all from Invitrogen) and serum starved overnight in high-glucose Dulbecco's modified Eagle medium (DMEM) medium, 1% glutamine-penicillin-streptomycin and 1% sodium pyruvate (all from Invitrogen). The cells were then incubated with fresh serum-free medium (high-glucose Dulbecco's modified Eagle medium (DMEM) medium, 1% glutamine-penicillin-streptomycin and 1% sodium pyruvate (all from Invitrogen)) for another hour.

The medium was then replaced with fresh serum-free medium, medium containing 0.1 μg/ml exosomes prepared as previously described in Lai, R. C., Arslan, F., Lee, M. M., Sze S. K., Choo, A., Chen, T. S., Salto-Tellez, M., Timmers, L., Lee, C. N., El Oakley, R. M., Pasterkamp, G., de Kleijn, D. P. V., Lim S.-K.† (2010) Exosome secreted by MSC reduces myocardial ischemia/reperfusion injury. Stem Cell Research 4: 214-222. with 100, 10, 1 or 0.1 μM AMP.

After 5 minutes, the cells were harvested, lysed and analysed by western blot hybridization.

10 μg total proteins were immunoblotted using 1:2000 dilution of rabbit anti-pERK (Santa Cruz Biotechnology Inc) 1/2, 1:2000 dilution of rabbit anti ERK1/2 (Santa Cruz Biotechnology Inc), 1:500 dilution rabbit anti-pAKT (Santa Cruz Biotechnology Inc) or 1:500 dilution of rabbit anti AKT (Santa Cruz Biotechnology Inc).

Results

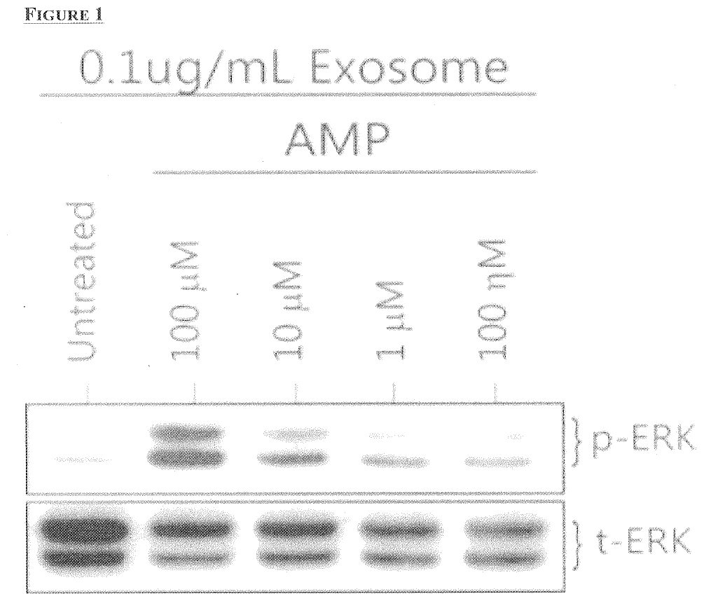

The results are shown in FIG. 1.

As FIG. 1 shows, MSC exosome through CD73 BC065937.1-mediated hydrolysis of AMP induced ERK (ERK1—NM_001040056.2; ERK2—NM_138957.2) and AKT NM_005163.2 pro-survival signaling by adenosine. Furthermore, this induction is proportional to the concentration of AMP over a 3-log concentration range of AMP.

### Example 2. MSC Exosomes Through CD73-Mediate Hydrolysis of AMP Induced ERK and AKT Pro-Survival Signaling by Adenosine—Increasing MSC Exosome Concentration

Materials and Methods

H9C2 cardiomyocytes (ATCC) were plated onto a 6 well plate at 200,000 cells per well and serum starved overnight.

The cells were then incubated with fresh serum-free medium for another hour. The medium was then replaced with fresh serum-free medium, medium containing 100 μM AMP (Sigma Aldrich) with 1.0, 0.1 or 0.01 μg/ml exosomes prepared as previously described in Lai, R. C., Arslan, F., Lee, M. M., Sze S. K., Choo, A., Chen, T. S., Salto-Tellez, M., Timmers, L., Lee, C. N., El Oakley, R. M., Pasterkamp, G., de Kleijn, D. P. V., Lim S.-K.† (2010) Exosome secreted by MSC reduces myocardial ischemia/reperfusion injury. Stem Cell Research 4: 214-222.

After 5 minutes, the cells were harvested, lysed and analysed by western blot hybridization.

10 μg total proteins were immunoblotted using 1:2000 dilution of rabbit anti-pERK (Santa Cruz Biotechnology Inc) 1/2, 1:2000 dilution of rabbit anti ERK1/2 (Santa Cruz Biotechnology Inc), 1:500 dilution rabbit anti-pAKT (Santa Cruz Biotechnology Inc) or 1:500 dilution of rabbit anti AKT (Santa Cruz Biotechnology Inc).

Results

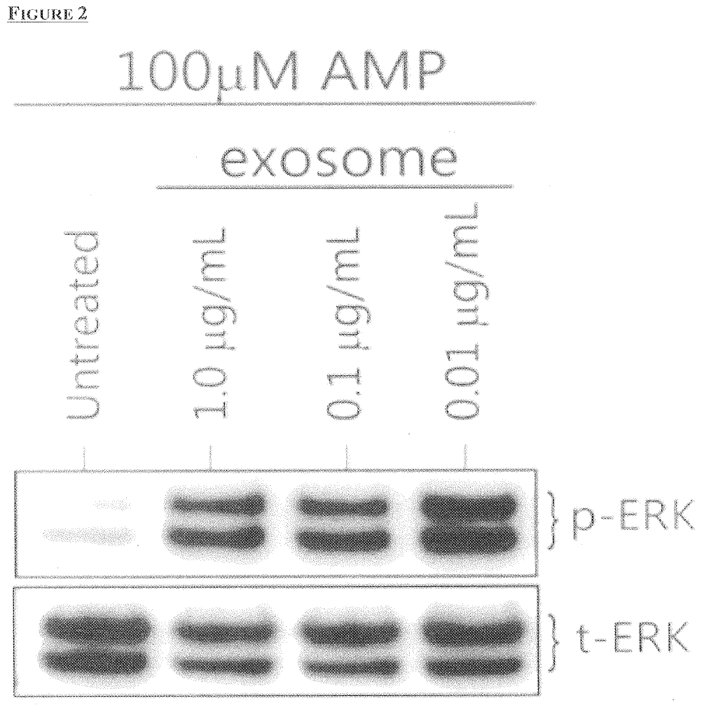

The results are shown in FIG. 2.

FIG. 2 shows that increasing MSC exosome concentration over a 3-log concentration range in the presence of constant AMP concentration did not result in a significant variation in the induction of ERK and AKT pro-survival signalling.

### Example 3. Discussion for Example 1 and Example 2—Cell-Death Homeostasis

The observation that MSC exosomes can induce a proportional adenosine-mediated response over a 3-log concentration range of AMP but not over a 3-log concentration range of exosome suggests that this activity is dependent on the severity of injury and is efficacious over an extensive range of injury. Importantly, the resolution of the injury as represented by decreasing concentration of AMP resulted in proportionately reduced adenosine signaling that is independent of exosome concentration. Practically, this greatly mitigates the risk of under- or over-dosing.

The other advantage of this system in moderating a pro-apoptotic microenvironment is the time lag between the pro-death signaling by ATP and the pro-survival signaling by adenosine caused by the time taken to hydrolyse ATP to AMP and then AMP to adenosine. As a consequence of this time lag, MSC exosome does not interfere with the pro-death activity of extracellular ATP necessary for the attack and defence mechanism of the organism but position its activity to minimize any ensuing collateral damage or expedite the restoration of cell homeostasis.

In summary, MSC exosomes could mount an adenosine pro-survival response that is delayed but proportionate to an ATP-mediated pro-death signal to facilitate a restoration of a homeostatic equilibrium in cell proliferation and death.

### Example 4. MSC Exosomes and Allogeneic Skin Graft Survival

Materials and Methods

Tail skin from C57BL/6 mice (A*STAR Biological Resource Center) was grafted onto BALB/c mice (A*STAR Biological Resource Center). 0.3 μg exosomes in 50 μl PBS as previously described in Lai, R. C., Arslan, F., Lee, M. M., Sze S. K., Choo, A., Chen, T. S., Salto-Tellez, M., Timmers, L., Lee, C. N., El Oakley, R. M., Pasterkamp, G., de Kleijn, D. P. V., Lim S.-K.† (2010) Exosome secreted by MSC reduces myocardial ischemia/reperfusion injury. Stem Cell Research 4: 214-222 or 50 μl PBS were injected subcutaneously into each recipient mouse every day for 4 days and then every other day for 15 days.

At day 7 when the dressing was removed, graft was scored for rejection every two days and photographed every other day.

Two independent experiments, each consisting of ten grafted and ten non-grafted mice in exosome-treated group, and ten grafted and ten non-grafted mice in PBS-treated group were performed. The mean rejection score over time was determined.

Each data point represented the mean with SEM. P value was determined by ANOVA.

Results

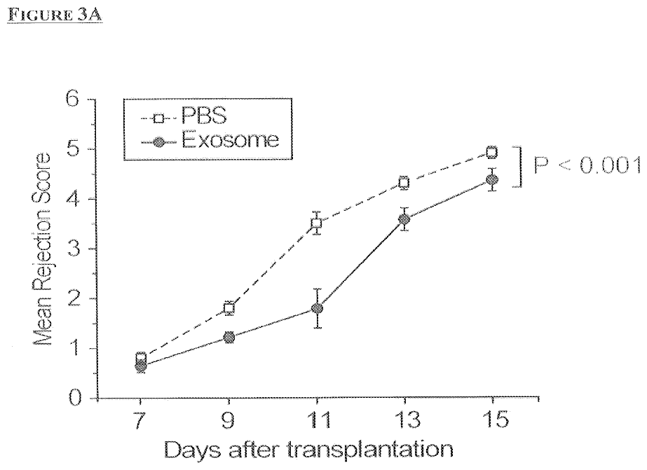

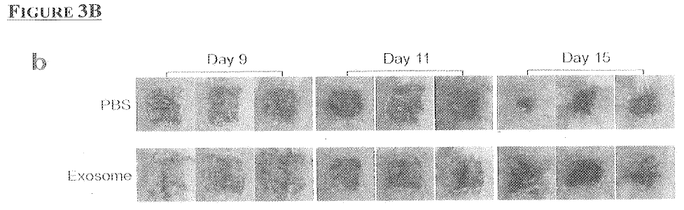

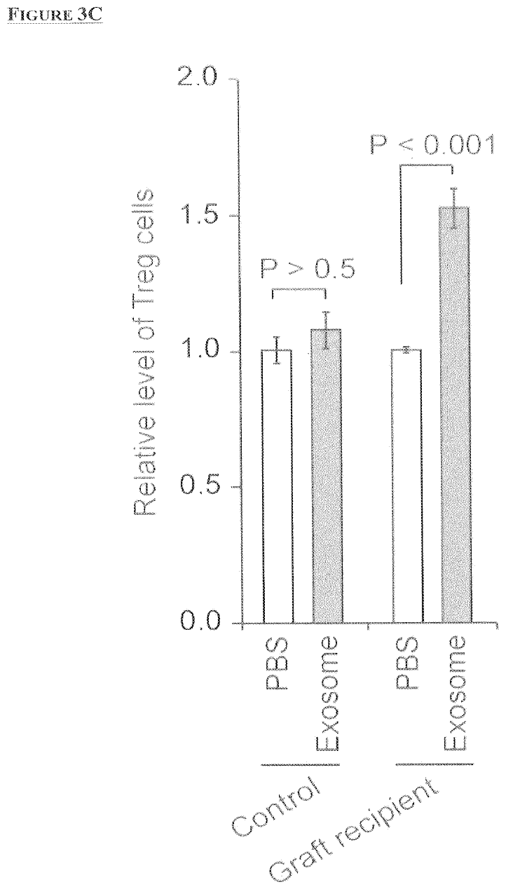

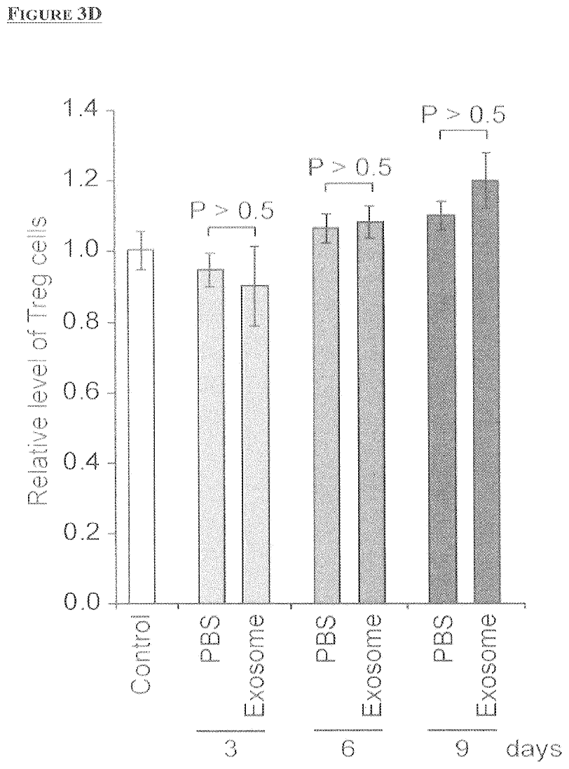

The results are shown in FIG. 3A, FIG. 3B, FIG. 3C and FIG. 3D.

FIG. 3B shows representative skin allograft in PBS or MSC exosome-treated mice at day 9, 11 and 15 after grafting.

FIG. 3C shows tregs in spleens of PBS or MSC exosome-treated mice. Fifteen days after grafting, splenocytes were purified from PBS or MSC exosome-treated mice, and stained for CD4, CD25 and Foxp3. The Treg levels were normalized to that of PBS vehicle control and presented as mean (±SD) of triplicate samples.

FIG. 3D shows an intrasplenic injection experiment. MSC exosomes were injected directly into the spleen at a dosage of 0.3 μg/mouse, and PBS as a vehicle control. After 3, 6 and 9 days, the spleens were respectively isolated from PBS or MSC exosome administrated mice for further FACS analysis of CD4+CD25+Foxp3+ Treg differentiation. Data was normalized to the untreated control and presented as mean (±SD) of triplicate samples.

### Example 5. Discussion for Example 4—Immune Homeostasis

Immune homeostasis refers to physiological equilibrium between “attack and defence” mechanism against pathogens, foreign or diseased tissues, and “tolerance” for self. This equilibrium is maintained by checks and balances involving many immune cell types and soluble mediators. Dysregulation of immune homeostasis compromises the body's defence and self-nonself recognition leading to disease or autoimmunity.

We have previously demonstrated that in vitro, MSC exosomes induce Treg polarization through the activation of monocytes (International Patent Publication WO 2012/108842).

When BALB/cJ mice were grafted with mice tail skin from C57BL/6J mice and treated with subcutaneous injections of 0.3 μg exosomes in 50 μl PBS or 50 μl PBS, exosome- and PBS-treated mice took 13 and 11 days to reject the grafts, respectively (FIG. 3A and FIG. 3B).

The level of Tregs was significantly higher in the spleens of exosome-treated graft recipient animals versus the saline-treated animals (P=0.002, FIG. 3C).

Interestingly, Treg induction was not observed in the spleens of non-graft recipient mice that had the same exosome treatment regimen with exosomes (P>0.5; FIG. 3C). Intrasplenic injection of exosomes or PBS into mice also did not induce a higher Treg level in exosome-treated animals (P>0.5; FIG. 3D).

Therefore, MSC exosome induce Tregs only when immune system is activated. Since Tregs are immunosuppressive, the induction of Tregs by MSC exosomes in an activated immune system demonstrated that MSC exosomes can attenuate immune activity and enhanced the restoration of immune homeostasis. Conversely, as MSC exosomes did not elicit Treg induction in mice that have not been immunologically challenged, MSC exosomes would not induce immune suppression and disrupt immune homeostasis.

In summary, MSC exosomes induce expansion of Tregs in animals whose immune activity is stimulated. In animals without immune stimulation, MSC exosomes have no effect on Treg expansion.

Therefore, MSC exosomes could facilitate immune homeostasis by attenuating reactivity through Treg expansion and when immune homeostasis is restored, this effect on Treg expansion dissipates.

### Example 6. Cell Adhesion Homeostasis

To demonstrate the capacity to restore cell adhesion homeostasis, a confluent MSC culture was trypsinized to form a single cell suspension. After neutralization and centrifugation, the trypsinized cells were re-suspended in growth medium. Equal volume of the cell suspension was plated on tissue culture plates that had been pre-coated with equal volumes of coating medium i.e. PBS, PBS with 6.25, 25 and 200 μg/ml MSC exosomes or 1 mg/ml gelatine solution. At 2 and 24 hours, the cultures were observed under the microscope.

In more detail, cell adhesion homeostasis in MSCs were disrupted by trypsinization as previously described (Lian, Lye et al. 2007).

The trypsinized single-cell MSC suspension was plated on gelatine- or MSC exosome-coated plates. The cell cultures were monitored at 2 and 24 hours for the establishment of cell adhesion to the plate.

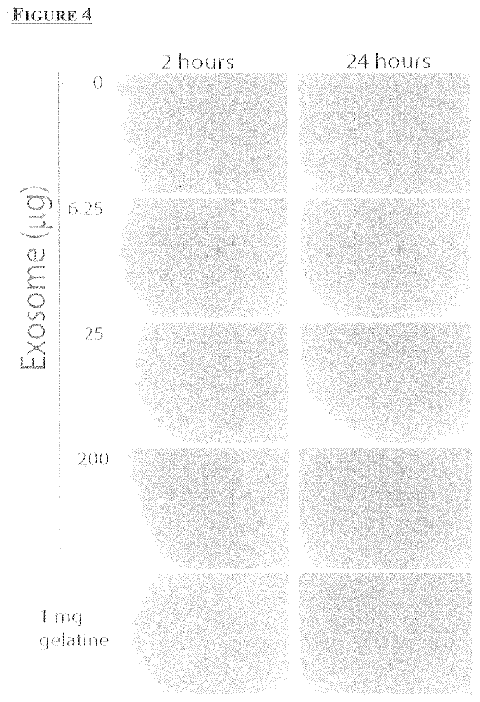

Within two hours, >90% of the cells plated on exosome- but not gelatine-coated plates have adhered to the plates signifying that MSC exosomes help restored cell adhesion state of cells to the state prior to trypsinization (FIG. 4).

### Example 7. Therapeutic Efficacy of Mesenchymal Stem Cell Exosomes Against Duchenne Muscular Dystrophy (DMD) in a Mouse Model

Background

Based on the capacity of MSC exosome to restore cell, immune and cell adhesion homeostasis, Mesenchymal Stem Cell (MSC) exosomes could be used to treat diseases where the pathology includes secondary immune reactivity. Such diseases include chronic heart failures (Fildes et al., 2009), atheriosclerosis (Koltsova et al., 2012) coronary and cerebral artery diseases (Stöllberger and Finsterer, 2002), neurodegenerative diseases (Amor et al., 2010), DMD (Duchenne Muscular Dystrophy) (Evans et al., 2009), and gerontological conditions (Agrawal et al., 2011, Vasto and Caruso, 2004) that are exacerbated by immune disorders such as chronic inflammation and reduced immune recognition of self versus non-self.

Method

The efficacy of MSC exosomes was evaluated in a mouse model of Duchenne Muscular Dystrophy. Briefly, sixteen (16) 4 weeks old hemizygous C57BL/10ScSn-Dmdmdx/J male mice were randomized by body weight into two treatment groups of 8 mice each, and dosed with either vehicle or exosomes (4 μg) intraperitoneally every two days for thirty two days. Clinical observation and body weights were completed twice a week. Forearm grip strength test was performed once weekly for a total of 5 times. On study day 35, mice were euthanized, plasma was prepared, and muscle tissue including extensor digitorum longus, tibialis anterior, diaphragm, and soleus were harvested and fixed in neutral buffered formalin. Plasma from each mouse was assayed for 32 mouse cytokines or chemokines (Eotaxin, G-CSF, GM-CSF, IFNγ, IL-1α, IL-1β, IL-2, IL-3, IL-4, IL-5, IL-6, IL-7, IL-9, IL-10, IL-12 (p40), IL-12 (p′70), IL-13, IL-15, IL-17A, IP-10, KC, LIF, LIX, MCP-1, M-CSF, MIG, MIP-1α, MIP-1β, MIP-2, RANTES, TNFα, VEGF) using multiplex bead technology. http://www.evetechnologies.com/discoveryAssayListMouse.php.

Results

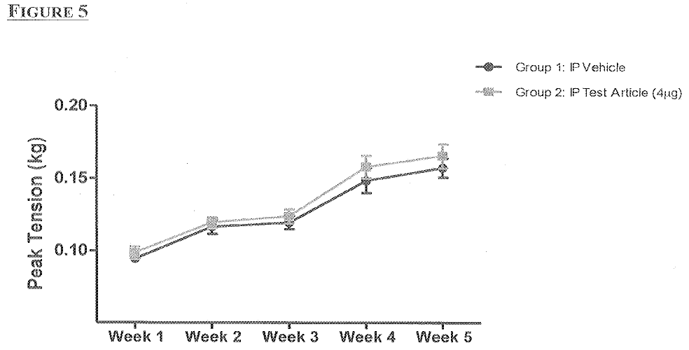

No significant difference was observed in the weight and grip strength of vehicle- and exosome-treated animals. However, the grip strength of vehicle-treated animals exhibited a trend of slower gain in grip strength over time relative to that in exosome-treated animals (FIG. 5).

The left and right muscle samples (extensor digitorum longus, tibialis anterior, diaphragm and soleus) of the mice histopathologically examined for evidence of dystropathology including atrophy, internalized nuclei, hypertrophy, degeneration/necrosis, and mineralization. The microscopic observations were graded with “0” being normal and “6” being severely pathological. Overall, vehicle-treated animals had a slightly higher total histology score when compared to exosome-treated animal (Table E1 below).

TABLE E1
The mean histology score for dystropathology in group
1 (vehicle-treated) and group 2 (exosome-treated) mice.

| Group: | 1 | 2 |
|---|---|---|
| Atrophy | 1.00 | 1.04 |
| Internalized nuclei | 2.59 | 2.39 |
| Hypertrophy | 0.47 | 0.46 |
| Degeneration/Necrosis | 1.44 | 1.32 |
| Mineralization | 0.00 | 0.11 |

Of the cytokines analysed, GM-CSF and IP10 were significantly elevated in exosome-treated animals (Table E2 below)

TABLE E2
Plasma from each mouse was assayed for 32 mouse cytokines or chemokines as listed below
using multiplex bead technology http://www.evetechnologies.com/discoveryAssayListMouse.php.
The value for each cytokine or chemokine is expressed as pg per ml plasma.

| | Eotaxin | | G-CSF | | GM-CSF | | IFNγ | | Il-1a | | Il-1b | | Il-2 | | Il-3 | |
|---|---|---|---|---|---|---|---|---|---|---|---|---|---|---|---|---|
| | Veh. | Exo | Veh. | Exo | Veh. | Exo | Veh. | Exo | Veh. | Exo | Veh. | Exo | Veh. | Exo | Veh | Exo |
| AVG | 810 | 729 | 178 | 134 | 41 | 5 | 0 | 0 | 274 | 891 | 20 | 71 | 10 | 14 | 0 | 1 |
| STDEV | 248 | 241 | 101 | 20 | 42 | 13 | 1 | 0 | 154 | 1651 | 33 | 95 | 13 | 11 | 0 | 3 |
| TTEST | 0.54 | | 0.26 | | 0.05 | | 0.35 | | 0.36 | | 0.21 | | 0.59 | | 0.36 | |
| | Il-4 | | Il-5 | | Il-6 | | Il-7 | | Il-9 | | Il-10 | | Il-12 (p40) | | Il-12 (p70) | |
| | Veh. | Exo | Veh. | Exo | Veh. | Exo | Veh. | Exo | Veh. | Exo | Veh. | Exo | Veh. | Exo | Veh. | Exo |
| AVG | 6 | 12 | 7 | 14 | 23 | 33 | 1 | 6 | 40 | 110 | 3 | 34 | 14 | 16 | 13 | 138 |
| STDEV | 2 | 19 | 7 | 7 | 12 | 14 | 2 | 6 | 19 | 89 | 6 | 53 | 16 | 21 | 24 | 218 |
| TTEST | 0.20 | | 0.06 | | 0.15 | | 0.09 | | 0.09 | | 0.18 | | 0.86 | | 0.18 | |
| | Il-13 | | Il-15 | | Il-17 | | IP-10 | | KC | | LIF | | LIX | | MCP-1 | |
| | Veh. | Exo | Veh. | Exo | Veh. | Exo | Veh. | Exo | Veh. | Exo | Veh. | Exo | Veh. | Exo | Veh. | Exo |
| AVG | 200 | 178 | 41 | 50 | 9 | 17 | 133 | 155 | 609 | 655 | 0 | 0 | 4191 | 7829 | 84 | 152 |
| STDEV | 50 | 79 | 30 | 53 | 6 | 15 | 20 | 14 | 295 | 552 | 0 | 0 | 3402 | 5442 | 25 | 87 |
| TTEST | 0.53 | | 0.68 | | 0.22 | | 0.03 | | 0.85 | | 0.64 | | 0.16 | | 0.09 | |
| | M-CSF | | MIG | | MIP-1a | | MIP-1b | | MIP-2 | | RANTES | | TNFa | | VEGF | |
| | Veh. | Exo | Veh. | Exo | Veh. | Exo | Veh. | Exo | Veh. | Exo | Veh. | Exo | Veh. | Exo | Veh. | Exo |
| AVG | 5 | 6 | 36 | 44 | 34 | 45 | 60 | 60 | 54 | 68 | 17 | 38 | 3 | 15 | 1 | 1 |
| STDEV | 1 | 1 | 8 | 9 | 17 | 34 | 40 | 21 | 29 | 40 | 13 | 37 | 3 | 15 | 0 | 1 |

| | | | | | | | | |
|---|---|---|---|---|---|---|---|---|
| TTEST | 0.38 | 0.13 | 0.45 | 0.97 | 0.47 | 0.19 | 0.09 | 0.06 |

(Veh.: vehicle-treated mice; Exo: exosome-treated mice; AVG: average; STDEV: Standard deviation; TTest: Student t-test)

GM-CSF has been shown to promote tissue healing after injury such as in colonic mucosal healing, wound healing (Bernasconi et al., 2010) (Mann et al., 2001, Lim et al., 2013, Hu et al., 2011, Groves and Schmidt-Lucke, 1999, Jorgensen et al., 2002) and bacterial infection (Steinwede et al., 2011).

IP 10 is important in the dissociation of new blood vessels that were generated during the regenerative phase of wound healing (Bodnar et al., 2009). This dissociation is essential to the formation of the avascular mature skin. IP-10 has also been shown to attenuate Bleomycin-Induced pulmonary fibrosis (Keane et al., 1999).

Conclusion

MSC exosomes improved muscle tissue pathology in DMD (Duchenne Muscular Dystrophy) mice and induced the production of cytokines known to enhance tissue repair.

### Example 8. Therapeutic Efficacy of Mesenchymal Stem Cell Exosomes in a Mouse Model of GVHD (Graft Versus Host Disease)

Background

Based on the capacity of MSC exosome to restore cell, immune and cell adhesion homeostasis, Mesenchymal Stem Cell (MSC) exosomes could be used to treat diseases where the pathology includes secondary immune reactivity. Such diseases include chronic heart failures (Fildes et al., 2009), atheriosclerosis (Koltsova et al., 2012) coronary and cerebral artery diseases (Stöllberger and Finsterer, 2002), neurodegenerative diseases (Amor et al., 2010), DMD (Duchenne Muscular Dystrophy) (Evans et al., 2009), and gerontological conditions (Agrawal et al., 2011, Vasto and Caruso, 2004) that are exacerbated by immune disorders such as chronic inflammation and reduced immune recognition of self versus non-self.

Method

The efficacy of MSC exosomes was evaluated in a mouse model of GVHD (Graft Versus Host Disease). Briefly, thirty (30) 6-8 week old female NSG (JAX Stock #005557; Jax Laboratory) mice were ear notched for identification and housed in individually and positively ventilated polysulfone cages with HEPA filtered air at a density of up to 5 mice per cage. Cages were changed every two weeks. The animal room was lighted entirely with artificial fluorescent lighting, with a controlled 12 h light/dark cycle (6 am to 6 pm light). The normal temperature and relative humidity ranges in the animal rooms were 22±4° C. and 50±15%, respectively. The animal rooms were set to have 15 air exchanges per hour. Filtered tap water, acidified to a pH of 2.5 to 3.0, and standard lab chow were provided ad libitum. After acclimating for 3-7 days, mice were grouped per body weight and irradiated with 100 RADS with an X-ray irradiator source on study day 0. Four hours post-irradiation, mice received 10×10 6 human PBMC injected intraperitoneally. Mice were dosed with vehicle, exosome (10 μg) and Enbrel (100 μg) injected intraperitoneally on day 1, 4, 7, 10 . . . up to day 55. Clinical observations and body weight measurement were performed three times weekly. Mice were euthanized by CO2 asphyxiation before final study take down on Day 50 if they show any of the following:

a) >20% weight loss from their starting weight,

b) cold to touch,

c) lethargic, hunched posture and scruffy coat

Results

At day 55, there were three survivors in the vehicle group, 6 survivors in the exosome group and 4 survivors in the Enbrel group.

Conclusion

MSC exosome enhanced survival of mice in GVHD (Graft Versus Host Disease).

### Example 9. Therapeutic Efficacy of Mesenchymal Stem Cell Exosomes in Epidermolysis Bullosa (EB)

The most severe forms of epidermolysis bullosa are the junctional EBs (JEBs) or the dystrophic EBs (DEB).

Dystrophic epidermolysis bullosa (DEB) is caused by mutations in COL7A1 gene resulting reduction or absence of C7 collagen, or truncation of C7 collagen. Therefore, the therapeutic target in the treatment of DEB is to correct for this genetic defect by either gene therapy to replace or deliver a functional COL7A1 gene, or by administering C7 collagen (Vanden Oever et al 2014). While gene therapy is advantageous in that it could potentially provide a permanent resolution of the C7 collagen deficiency, the appropriate strategy to deliver the gene to enough target cells remains challenging. In comparison, the administration of C7 collagen is relatively simple and could correct the disease phenotype in mice (Remington et al 2009).

Mesenchymal stem cells have been shown to alleviate RDEB disease phenotype in Col7a1-null mice by secreting collagen 7 which is deposited at dermal-epidermal junction (Alexeev et al 2011). Together these studies implied that collagen 7-containing secretion from mesenchymal stem cells could alleviate RDEB disease phenotype.

Protocol

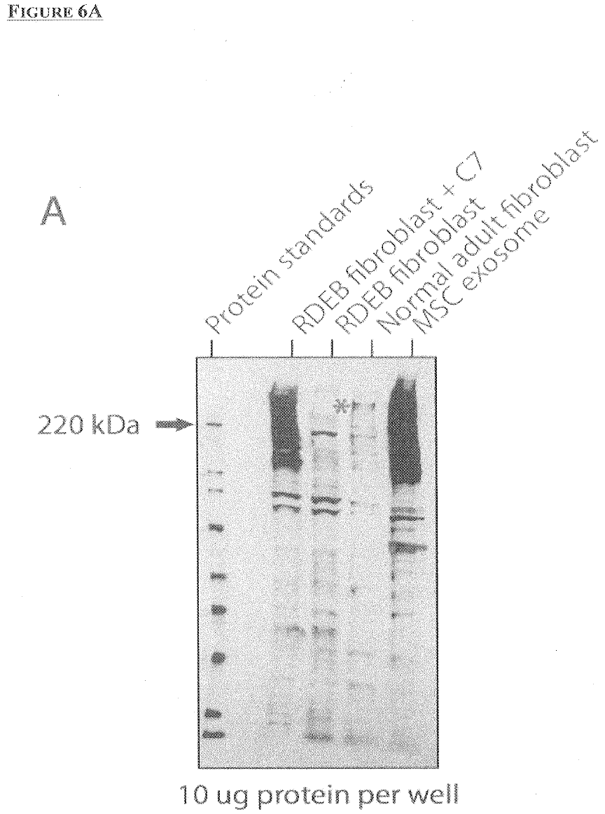

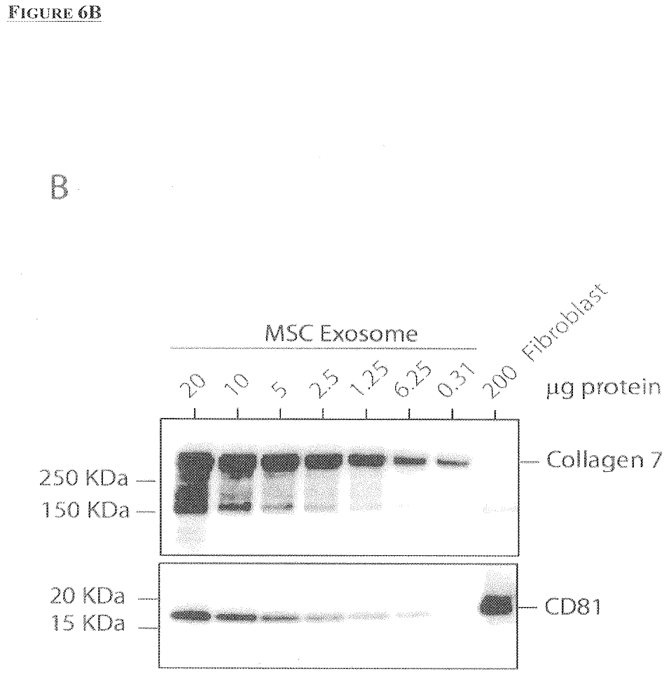

We conducted a Western blot analysis of cell or exosome lysates for collagen 7, shown in FIG. 6A.

Lanes from left to right show molecular weight markers, RDEB human dermal fibroblasts over-expressing collagen 7, RDEB human dermal fibroblasts, normal human dermal fibroblasts and human mesenchymal stem cell (MSC) exosome.

The fibroblast lysate was prepared by removing the culture medium from a confluent dish of primary human dermal fibroblasts and adding a cell lysate buffer to the dish. MSC exosomes were prepared as previously described6.

The fibroblast and exosome lysates w200 ere analysed by a standard immunoblotting assay using an Anti-Collagen VII antibody [LH7.2] (ab6312) from Abcam. * indicates collagen 7.

We also estimated the concentration of collagen 7 in MSC exosomes relative to that in normal human dermal fibroblast. The results are shown in FIG. 6B.

A two-fold serial dilution of MSC exosome lysate starting with 20 μg protein was prepared and loaded in parallel with 200 μg fibroblast lysate. The blot was also probed for CD81 as a loading control.

Results

The results are shown in FIG. 6A and FIG. 6B.

We observed that most of the collagen 7 secreted by mesenchymal stem cells are associated with exosomes (FIG. 6A and FIG. 6B).

The amount of collagen per μg exosome protein is more 100 times more than that produced by 1 μg normal human fibroblast lysate. Since dermal injections of allogeneic fibroblasts have been shown to be safe, and have the potential to improve skin strength and reduce blistering by increasing the deposit of collagen 7 at the dermal-epidermal junction (Wong et al 2008) and injection of collagen 7 alone could alleviate RDEB (Woodley et al., 2004, Remington et al., 2008), the administration of exosome which is highly enriched in collagen 7 would alleviate RDEB phenotype.

### Example 10. Restoration of Immune Homeostasis by Mesenchymal Stem Cell Exosomes in Graft Versus Host Disease (GVHD)

This Example demonstrates the use of mesenchymal stem cell exosomes in restoring immune homeostasis is in Graft versus Host Disease (GVHD).

We used a well established mouse model of delayed lymphocyte infusion (DLI) model described in Choi, J., et al. IFNgammaR signaling mediates alloreactive T-cell trafficking and GVHD. Blood 120, 4093-4103 (2012) and Rettig, M. P., et al. Kinetics of in vivo elimination of suicide gene-expressing T cells affects engraftment, graft-versus-host disease, and graft-versus-leukemia after allogeneic bone marrow transplantation. J Immunol 173, 3620-3630 (2004).

Recipient Balb/c mice were irradiated with 900 cGy on day −1 and transplanted with 5×106 TCD-BM from donor B6 CD45.1+ mice on day 0.

On day 11 they are infused with 2×106 donor B6 CD45.2+ T cells. On day 12 post transplant (i.e. one day after the DLI) until day 32, 0.8 μg exosomes or PBS were administered to the mice every other day.

At day 60, all surviving mice were sacrificed.

Results

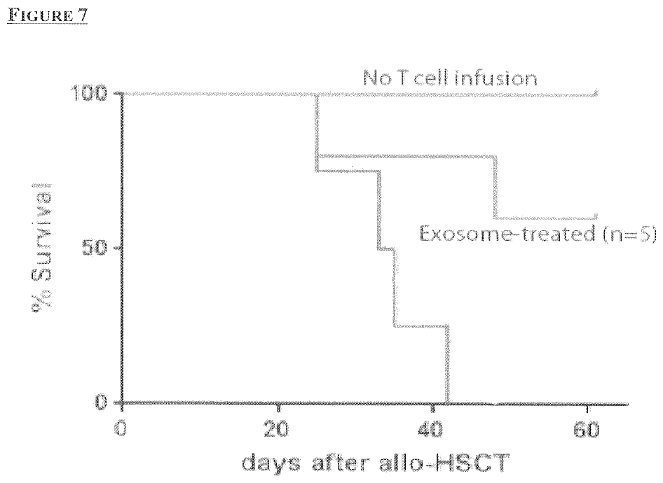

The results are shown in FIG. 7, which shows a survival curve of DLI mice.

FIG. 7 demonstrates that survival of exosome-treated mice was significantly higher than PBS-treated animals (p=0.0368).

### Example 11. Enhancement of Cell Adhesion and Therapeutic Effects of Mesenchymal Stem Cell Exosomes in Epidermolysis Bullosa (EB)

This Example demonstrates the use of mesenchymal stem cell exosomes in enhancing cell adhesion in vivo and exertion of therapeutic effects in epidermolysis bullosa (EB).

Mesenchymal stem cell derived exosomes were administered intraperitoneally to a hypomorphic mouse model of dystrophic epidermolysis bullosa (described in Fritsch, A., S. Loeckermann, et al. (2008). “A hypomorphic mouse model of dystrophic epidermolysis bullosa reveals mechanisms of disease and response to fibroblast therapy.” The Journal of Clinical Investigation 118(5): 1669-1679).

The mice were dosed at 25 or 50 microgram per pup every two days shortly after birth.

Results

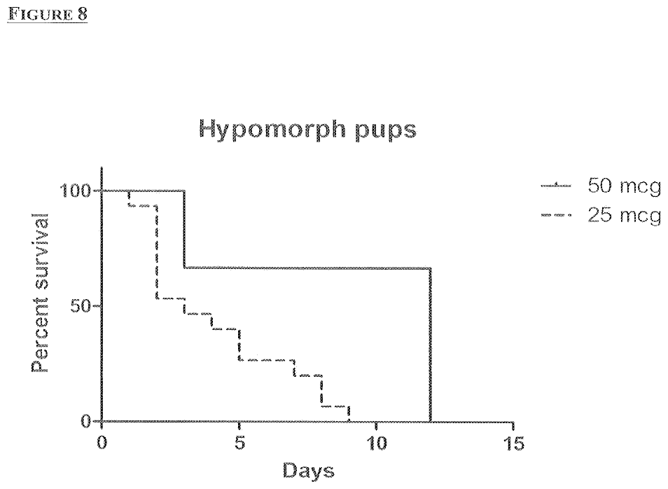

The results are shown in FIG. 8, which is a survival curve of exosome-treated hypomorphic mouse model of dystrophic epidermolysis bullosa.

As shown in FIG. 8, survival curves between mice given 25 or 50 microgram were significantly different based on log-rank test P=0.0333.

### REFERENCES

1. Arslan, F., et al. Mesenchymal stem cell-derived exosomes increase ATP levels, decrease oxidative stress and activate PI3K/Akt pathway to enhance myocardial viability and prevent adverse remodeling after myocardial ischemia/reperfusion injury. Stem Cell Res 10, 301-312 (2013).
2. Lai, R., et al. Mesenchymal Stem Cell Exosomes: The Future MSC-Based Therapy? in Mesenchymal Stem Cell Therapy (ed. Chase, L. G. V., Mohan C.) 39-62 (Humana Press, 2013).
3. Lai, R. C., Chen, T. S. & Lim, S. K. Mesenchymal stem cell exosome: a novel stem cell-based therapy for cardiovascular disease. Regenerative medicine 6, 481-492 (2011).
4. Lai, R. C., et al. Proteolytic Potential of the MSC Exosome Proteome: Implications for an Exosome-Mediated Delivery of Therapeutic Proteasome. Int J Proteomics 2012, 971907 (2012).
5. Lai, R. C., Yeo, R. W., Tan, K. H. & Lim, S. K. Mesenchymal stem cell exosome ameliorates reperfusion injury through proteomic complementation. Regenerative medicine 8, 197-209 (2013).
6. Lai, R. C., et al. Mesenchymal Stem Cell Exosomes: The Future MSC-based Therapy? in Mesenchymal Stem Cell Therapy (eds. Chase, L. G. & Vemuri, M. C.) (Humana Press, 2012).
7. Luthje, J. Origin, metabolism and function of extracellular adenine nucleotides in the blood. Klin Wochenschr 67, 317-327 (1989).
8. Di Virgilio, F. Purines, Purinergic Receptors, and Cancer. Cancer Research 72, 5441-5447 (2012).
9. Aymeric, L., et al. Tumor Cell Death and ATP Release Prime Dendritic Cells and Efficient Anticancer Immunity. Cancer Research 70, 855-858 (2010).
10. Vitiello, L., Gorini, S., Rosano, G. & la Sala, A. Immunoregulation through extracellular nucleotides. Blood 120, 511-518 (2012).
11. Wan, J., Ristenpart, W. D. & Stone, H. A. Dynamics of shear-induced ATP release from red blood cells. Proceedings of the National Academy of Sciences (2008).
12. Boudreault, F. & Grygorczyk, R. Cell swelling-induced ATP release is tightly dependent on intracellular calcium elevations. The Journal of Physiology 561, 499-513 (2004).
13. Ayna, G., et al. ATP Release from Dying Autophagic Cells and Their

Phagocytosis Are Crucial for Inflammasome Activation in Macrophages. PLoS ONE 7, e40069 (2012).
14. Chekeni, F. B., et al. Pannexin 1 channels mediate/find-me signal release and membrane permeability during apoptosis. Nature 467, 863-867 (2010).
15. Luthje, J. & Ogilvie, A. Catabolism of Ap4A and Ap3A in whole blood. The dinucleotides are long-lived signal molecules in the blood ending up as intracellular ATP in the erythrocytes. Eur J Biochem 173, 241-245 (1988).
16. Colgan, S. P., Eltzschig, H. K., Eckle, T. & Thompson, L. F. Physiological roles for ecto-5′-nucleotidase (CD73). Purinergic Signal 2, 351-360 (2006).
17. Yellon, D. M. & Baxter, G. F. Reperfusion Injury Revisited: Is There a Role for Growth Factor Signaling in Limiting Lethal Reperfusion Injury? Trends in Cardiovascular Medicine 9, 245-249 (1999).
18. Hausenloy, D. J. & Yellon, D. M. New directions for protecting the heart against ischaemia-reperfusion injury: targeting the Reperfusion Injury Salvage Kinase (RISK)-pathway. Cardiovasc Res 61, 448-460 (2004).
19. Fildes, J. E., Shaw, S. M., Yonan, N. & Williams, S. G. The Immune System and Chronic Heart Failure: Is the Heart in Control? Journal of the American College of Cardiology 53, 1013-1020 (2009).
20. Koltsova, E. K., et al. Dynamic T cell-APC interactions sustain chronic inflammation in atherosclerosis. The Journal of Clinical Investigation 122, 3114-3126 (2012).
21. Stöllberger, C. & Finsterer, J. Role of Infectious and Immune Factors in Coronary and Cerebrovascular Arteriosclerosis. Clinical and Diagnostic Laboratory Immunology 9, 207-215 (2002).
22. Amor, S., Puentes, F., Baker, D. & Van Der Valk, P. Inflammation in neurodegenerative diseases. Immunology 129, 154-169 (2010).
23. Evans, N. P., Misyak, S. A., Robertson, J. L., Bassaganya-Riera, J. & Grange, R. W. Immune-Mediated Mechanisms Potentially Regulate the Disease Time-Course of Duchenne Muscular Dystrophy and Provide Targets for Therapeutic Intervention. PM&R 1, 755-768 (2009).
24. Agrawal, A., Sridharan, A., Prakash, S. & Agrawal, H. Dendritic cells and aging: consequences for autoimmunity. Expert Review of Clinical Immunology 8, 73-80 (2011).
25. Vasto, S. & Caruso, C. Immunity & Ageing: a new journal looking at ageing from an immunological point of view. Immun Ageing 1, 1 (2004).
Bellayr, I., X. Mu, et al. (2009). “Biochemical insights into the role of matrix metalloproteinases in regeneration: challenges and recent developments.” Future Med Chem 1(6): 1095-1111.
Cagnoni, M. L., I. Ghersetich, et al. (1994). “Treatment of psoriasis vulgaris with topical calcipotriol: is the clinical improvement of lesional skin related to a down-regulation of some cell adhesion molecules?” Acta Derm Venereol Suppl (Stockh) 186: 55-57.
Chan, T.-F., A. Poon, et al. (2008). “Natural variation in four human collagen genes across an ethnically diverse population.” Genomics 91(4): 307-314.
Clark, I. M., T. E. Swingler, et al. (2008). “The regulation of matrix metalloproteinases and their inhibitors.” Int J Biochem Cell Biol 40(6-7): 1362-1378.
De Bleecker, J. L. and A. G. Engel (1994). “Expression of cell adhesion molecules in inflammatory myopathies and Duchenne dystrophy.” J Neuropathol Exp Neurol 53(4): 369-376.
Deichmann, M., H. Kurzen, et al. (2003). “Adhesion molecules CD171 (L1CAM) and CD24 are expressed by primary neuroendocrine carcinomas of the skin (Merkel cell carcinomas).” J Cutan Pathol 30(6): 363-368.
Frantz, C., K. M. Stewart, et al. (2010). “The extracellular matrix at a glance.” J Cell Sci Suppl 123(24): 4195-4200.
Gnanapavan, S. and G. Giovannoni (2013). “Neural cell adhesion molecules in brain plasticity and disease.” Multiple Sclerosis and Related Disorders 2(1): 13-20.
Golias, C., E. Tsoutsi, et al. (2007). “Review. Leukocyte and endothelial cell adhesion molecules in inflammation focusing on inflammatory heart disease.” In Vivo 21(5): 757-769.
Gotts, J. E. and M. A. Matthay (2012). “Mesenchymal stem cells and the stem cell niche: a new chapter.” Am J Physiol Lung Cell Mol Physiol 302(11): L1147-1149.
Halper, J. and M. Kjaer (2014). “Basic components of connective tissues and extracellular matrix: elastin, fibrillin, fibulins, fibrinogen, fibronectin, laminin, tenascins and thrombospondins.” Adv Exp Med Biol 802: 31-47.
Hellewell, P. G. (1993). “Cell adhesion molecules and potential for pharmacological intervention in lung inflammation.” Pulm Pharmacol 6(2): 109-118.
Hillis, G. S. and A. D. Flapan (1998). “Cell adhesion molecules in cardiovascular disease: a clinical perspective.” Heart 79(5): 429-431.
Jang, Y., A. M. Lincoff, et al. (1994). “Cell adhesion molecules in coronary artery disease.” J Am Coll Cardiol 24(7): 1591-1601.
Järveläinen, H., A. Sainio, et al. (2009). “Extracellular Matrix Molecules: Potential Targets in Pharmacotherapy.” Pharmacological Reviews 61(2): 198-223.
Johnson, J. P. (1991). “Cell adhesion molecules of the immunoglobulin supergene family and their role in malignant transformation and progression to metastatic disease.” Cancer Metastasis Rev 10(1): 11-22.
Johnson, J. P. (1992). “Cell adhesion molecules in neoplastic disease.” Int J Clin Lab Res 22(2): 69-72.
Kielty, C. M., M. J. Sherratt, et al. (2002). “Elastic fibres.” J Cell Sci 115(Pt 14): 2817-2828.
Kim, S. H., J. Turnbull, et al. (2011). “Extracellular matrix and cell signalling: the dynamic cooperation of integrin, proteoglycan and growth factor receptor.” J Endocrinol 209(2): 139-151.
Lian, Q., E. Lye, et al. (2007). “Derivation of clinically compliant MSCs from CD105+, CD24− differentiated human ESCs.” Stem Cells 25(2): 425-436.
Lu, P., K. Takai, et al. (2011). “Extracellular matrix degradation and remodeling in development and disease.” Cold Spring Harb Perspect Biol 3(12).
Mishra, L., B. B. Mishra, et al. (1993). “In vitro cell aggregation and cell adhesion molecules in Crohn's disease.” Gastroenterology 104(3): 772-779.
Nakamura, S., H. Ohtani, et al. (1993). “In situ expression of the cell adhesion molecules in inflammatory bowel disease. Evidence of immunologic activation of vascular endothelial cells.” Lab Invest 69(1): 77-85.
Patino, M. G., M. E. Neiders, et al. (2002). “Collagen: An Overview.” Implant Dentistry 11(3): 280-285.
Ricard-Blum, S. (2011). “The collagen family.” Cold Spring Harb Perspect Biol 3(1): a004978.
Schiller, B. and G. Elinder (1999). “Inflammatory parameters and soluble cell adhesion molecules in Swedish children with Kawasaki disease: relationship to cardiac lesions and intravenous immunoglobulin treatment.” Acta Paediatr 88(8): 844-848.
Schofield, R. (1978). “The relationship between the spleen colony-forming cell and the haemopoietic stem cell.” Blood Cells 4(1-2): 7-25.
Schuermann, G. M., A. E. Aber-Bishop, et al. (1993). “Altered expression of cell adhesion molecules in uninvolved gut in inflammatory bowel disease.” Clin Exp Immunol 94(2): 341-347.
Tkachenko, E., J. M. Rhodes, et al. (2005). “Syndecans: New Kids on the Signaling Block.” Circulation Research 96(5): 488-500.
Ukkonen, M., K. Wu, et al. (2007). “Cell surface adhesion molecules and cytokine profiles in primary progressive multiple sclerosis.” Mult Scler 13(6): 701-707.
Villanacci, V., F. Facchetti, et al. (1993). “Expression of cell adhesion molecules in jejunum biopsies of children with coeliac disease.” Ital J Gastroenterol 25(3): 109-116.
Vora, A. J., D. Kidd, et al. (1997). “Lymphocyte-endothelial cell interactions in multiple sclerosis: disease specificity and relationship to circulating tumour necrosis factor-alpha and soluble adhesion molecules.” Mult Scler 3(3): 171-179.
Wegner, C. D., R. H. Gundel, et al. (1992). “Expression and probable roles of cell adhesion molecules in lung inflammation.” Chest 101(3 Suppl): 34S-39S.
Kopecki Z, Murrell D F, Cowin A J. Raising the roof on epidermolysis bullosa (EB): a focus on new therapies. Wound Practice & Research: Journal of the Australian Wound Management Association. 2009; 17(2): 76.
Vanden Oever M J, Tolar J. Advances in understanding and treating dystrophic epidermolysis bullosa. F1000Prime Rep. 2014; 6: 35.
Remington J, Wang X, Hou Y, Zhou H, Burnett J, Muirhead T, et al. Injection of recombinant human type VII collagen corrects the disease phenotype in a murine model of dystrophic epidermolysis bullosa. Mol Ther. 2009; 17(1): 26-33.
Alexeev V, Uitto J, Igoucheva O. Gene expression signatures of mouse bone marrow-derived mesenchymal stem cells in the cutaneous environment and therapeutic implications for blistering skin disorder. Cytotherapy. 2011; 13(1): 30-45.
Wong T, Gammon L, Liu L, Mellerio J E, Dopping-Hepenstal P J, Pacy J, et al. Potential of fibroblast cell therapy for recessive dystrophic epidermolysis bullosa. J Invest Dermatol. 2008; 128(9): 2179-89.
Lai R C, Arslan F, Lee M M, Sze N S, Choo A, Chen T S, et al. Exosome secreted by MSC reduces myocardial ischemia/reperfusion injury. Stem Cell Res. 2010; 4: 214-22.
AGRAWAL, A., SRIDHARAN, A., PRAKASH, S. & AGRAWAL, H. 2011. Dendritic cells and aging: consequences for autoimmunity. Expert Review of Clinical Immunology, 8, 73-80.
AMOR, S., PUENTES, F., BAKER, D. & VAN DER VALK, P. 2010. Inflammation in neurodegenerative diseases. Immunology, 129, 154-169.
BERNASCONI, E., FAVRE, L., MAILLARD, M. H., BACHMANN, D., PYTHOUD, C., BOUZOURENE, H., CROZE, E., VELICHKO, S., PARKINSON, J., MICHETTI, P. & VELIN, D. 2010. Granulocyte macrophage colony stimulating factor elicits bone marrow□ derived cells that promote efficient colonic mucosal healing. Inflammatory Bowel Diseases, 16, 428-441 10.1002/ibd.21072.
BODNAR, R. J., YATES, C. C., RODGERS, M. E., DU, X. & WELLS, A. 2009. IP-10 induces dissociation of newly formed blood vessels. J Cell Sci Suppl, 122, 2064-2077.
EVANS, N. P., MISYAK, S. A., ROBERTSON, J. L., BASSAGANYA-RIERA, J. & GRANGE, R. W. 2009. Immune-Mediated Mechanisms Potentially Regulate the Disease Time-Course of Duchenne Muscular Dystrophy and Provide Targets for Therapeutic Intervention. PM&R, 1, 755-768.
FILDES, J. E., SHAW, S. M., YONAN, N. & WILLIAMS, S. G. 2009. The Immune System and Chronic Heart Failure: Is the Heart in Control? Journal of the American College of Cardiology, 53, 1013-1020.
GROVES, R. W. & SCHMIDT-LUCKE, J. A. 1999. Recombinant human GM-CSF in the treatment of poorly healing wounds. Advances in skin & wound care, 13, 107-112.
HU, X., SUN, H., HAN, C., WANG, X. & YU, W. 2011. Topically applied rhGM-CSF for the wound healing: a systematic review. Burns, 37, 729-741.
JORGENSEN, L. N., AGREN, M. S., MADSEN, S. M., KALLEHAVE, F., VOSSOUGHI, F., RASMUSSEN, A. & GOTTRUP, F. 2002. Dose-dependent impairment of collagen deposition by topical granulocyte-macrophage colony-stimulating factor in human experimental wounds. Annals of surgery, 236, 684.
KEANE, M. P., BELPERIO, J. A., ARENBERG, D. A., BURDICK, M. D., XU, Z. J., XUE, Y. Y. & STRIETER, R. M. 1999. IFN-γ-Inducible Protein-10 Attenuates Bleomycin-Induced Pulmonary Fibrosis Via Inhibition of Angiogenesis. The Journal of Immunology, 163, 5686-5692.
KOLTSOVA, E. K., GARCIA, Z., CHODACZEK, G., LANDAU, M., MCARDLE, S., SCOTT, S. R., VON VIETINGHOFF, S., GALKINA, E., MILLER, Y. I., ACTON, S. T. & LEY, K. 2012. Dynamic T cell-APC interactions sustain chronic inflammation in atherosclerosis. The Journal of Clinical Investigation, 122, 3114-3126.
LIM, J.-Y., CHOI, B. H., LEE, S., JANG, Y. H., CHOI, J.-S. & KIM, Y.-M. 2013. Regulation of Wound Healing by Granulocyte-Macrophage Colony-Stimulating Factor after Vocal Fold Injury. PLoS ONE, 8, e54256.
MANN, A., BREUHAHN, K., SCHIRMACHER, P. & BLESSING, M. 2001. Keratinocyte-Derived Granulocyte-Macrophage Colony Stimulating Factor Accelerates Wound Healing: Stimulation of Keratinocyte Proliferation, Granulation Tissue Formation, and Vascularization. J Investig Dermatol, 117, 1382-1390.
STEINWEDE, K., TEMPELHOF, O., BOLTE, K., MAUS, R., BOHLING, J., UEBERBERG, B., LANGER, F., CHRISTMAN, J. W., PATON, J. C. & ASK, K. 2011. Local delivery of GM-CSF protects mice from lethal pneumococcal pneumonia. The Journal of Immunology, 187, 5346-5356.
STÖLLBERGER, C. & FINSTERER, J. 2002. Role of Infectious and Immune Factors in Coronary and Cerebrovascular Arteriosclerosis. Clinical and Diagnostic Laboratory Immunology, 9, 207-215.
VASTO, S. & CARUSO, C. 2004. Immunity & Ageing: a new journal looking at ageing from an immunological point of view. Immun Ageing, 1, 1.
1. Arslan, F., et al. Mesenchymal stem cell-derived exosomes increase ATP levels, decrease oxidative stress and activate PI3K/Akt pathway to enhance myocardial viability and prevent adverse remodeling after myocardial ischemia/reperfusion injury. Stem Cell Res 10, 301-312 (2013).
2. Lai, R., et al. Mesenchymal Stem Cell Exosomes: The Future MSC-Based Therapy? in Mesenchymal Stem Cell Therapy (ed. Chase, L. G. V., Mohan C.) 39-62 (Humana Press, 2013).
3. Lai, R. C., Chen, T. S. & Lim, S. K. Mesenchymal stem cell exosome: a novel stem cell-based therapy for cardiovascular disease. Regenerative medicine 6, 481-492 (2011).
4. Lai, R. C., et al. Proteolytic Potential of the MSC Exosome Proteome: Implications for an Exosome-Mediated Delivery of Therapeutic Proteasome. Int J Proteomics 2012, 971907 (2012).
5. Lai, R. C., Yeo, R. W., Tan, K. H. & Lim, S. K. Mesenchymal stem cell exosome ameliorates reperfusion injury through proteomic complementation. Regenerative medicine 8, 197-209 (2013).
6. Lai, R. C., et al. Mesenchymal Stem Cell Exosomes: The Future MSC-based Therapy? in Mesenchymal Stem Cell Therapy (eds. Chase, L. G. & Vemuri, M. C.) (Humana Press, 2012).
7. Luthje, J. Origin, metabolism and function of extracellular adenine nucleotides in the blood. Klin Wochenschr 67, 317-327 (1989).
8. Di Virgilio, F. Purines, Purinergic Receptors, and Cancer. Cancer Research 72, 5441-5447 (2012).
9. Aymeric, L., et al. Tumor Cell Death and ATP Release Prime Dendritic Cells and Efficient Anticancer Immunity. Cancer Research 70, 855-858 (2010).
10. Vitiello, L., Gorini, S., Rosano, G. & la Sala, A. Immunoregulation through extracellular nucleotides. Blood 120, 511-518 (2012).
11. Wan, J., Ristenpart, W. D. & Stone, H. A. Dynamics of shear-induced ATP release from red blood cells. Proceedings of the National Academy of Sciences (2008).
12. Boudreault, F. & Grygorczyk, R. Cell swelling-induced ATP release is tightly dependent on intracellular calcium elevations. The Journal of Physiology 561, 499-513 (2004).
13. Ayna, G., et al. ATP Release from Dying Autophagic Cells and Their Phagocytosis Are Crucial for Inflammasome Activation in Macrophages. PLoS ONE 7, e40069 (2012).
14. Chekeni, F. B., et al. Pannexin 1 channels mediate find-met signal release and membrane permeability during apoptosis. Nature 467, 863-867 (2010).
15. Luthje, J. & Ogilvie, A. Catabolism of Ap4A and Ap3A in whole blood. The dinucleotides are long-lived signal molecules in the blood ending up as intracellular ATP in the erythrocytes. Eur J Biochem 173, 241-245 (1988).
16. Colgan, S. P., Eltzschig, H. K., Eckle, T. & Thompson, L. F. Physiological roles for ecto-5′-nucleotidase (CD73). Purinergic Signal 2, 351-360 (2006).
17. Yellon, D. M. & Baxter, G. F. Reperfusion Injury Revisited: Is There a Role for Growth Factor Signaling in Limiting Lethal Reperfusion Injury? Trends in Cardiovascular Medicine 9, 245-249 (1999).
18. Hausenloy, D. J. & Yellon, D. M. New directions for protecting the heart against ischaemia-reperfusion injury: targeting the Reperfusion Injury Salvage Kinase (RISK)-pathway. Cardiovasc Res 61, 448-460 (2004).
19. Choi, J., et al. IFNgammaR signaling mediates alloreactive T-cell trafficking and GVHD. Blood 120, 4093-4103 (2012).
20. Rettig, M. P., et al. Kinetics of in vivo elimination of suicide gene-expressing T cells affects engraftment, graft-versus-host disease, and graft-versus-leukemia after allogeneic bone marrow transplantation. J Immunol 173, 3620-3630 (2004).
21. Fildes, J. E., Shaw, S. M., Yonan, N. & Williams, S. G. The Immune System and Chronic Heart Failure: Is the Heart in Control? Journal of the American College of Cardiology 53, 1013-1020 (2009).
22. Koltsova, E. K., et al. Dynamic T cell-APC interactions sustain chronic inflammation in atherosclerosis. The Journal of Clinical Investigation 122, 3114-3126 (2012).
23. Stöllberger, C. & Finsterer, J. Role of Infectious and Immune Factors in Coronary and Cerebrovascular Arteriosclerosis. Clinical and Diagnostic Laboratory Immunology 9, 207-215 (2002).
24. Amor, S., Puentes, F., Baker, D. & Van Der Valk, P. Inflammation in neurodegenerative diseases. Immunology 129, 154-169 (2010).
25. Evans, N. P., Misyak, S. A., Robertson, J. L., Bassaganya-Riera, J. & Grange, R. W. Immune-Mediated Mechanisms Potentially Regulate the Disease Time-Course of Duchenne Muscular Dystrophy and Provide Targets for Therapeutic Intervention. PM&R 1, 755-768 (2009).
26. Agrawal, A., Sridharan, A., Prakash, S. & Agrawal, H. Dendritic cells and aging: consequences for autoimmunity. Expert Review of Clinical Immunology 8, 73-80 (2011).
27. Vasto, S. & Caruso, C. Immunity & Ageing: a new journal looking at ageing from an immunological point of view. Immun Ageing 1, 1 (2004).
Bellayr, I., X. Mu, et al. (2009). “Biochemical insights into the role of matrix metalloproteinases in regeneration: challenges and recent developments.” Future Med Chem 1(6): 1095-1111.
Cagnoni, M. L., I. Ghersetich, et al. (1994). “Treatment of psoriasis vulgaris with topical calcipotriol: is the clinical improvement of lesional skin related to a down-regulation of some cell adhesion molecules?” Acta Derm Venereol Suppl (Stockh) 186: 55-57.
Chan, T.-F., A. Poon, et al. (2008). “Natural variation in four human collagen genes across an ethnically diverse population.” Genomics 91(4): 307-314.
Clark, I. M., T. E. Swingler, et al. (2008). “The regulation of matrix metalloproteinases and their inhibitors.” Int J Biochem Cell Biol 40(6-7): 1362-1378.
De Bleecker, J. L. and A. G. Engel (1994). “Expression of cell adhesion molecules in inflammatory myopathies and Duchenne dystrophy.” J Neuropathol Exp Neurol 53(4): 369-376.
Deichmann, M., H. Kurzen, et al. (2003). “Adhesion molecules CD171 (L1CAM) and CD24 are expressed by primary neuroendocrine carcinomas of the skin (Merkel cell carcinomas).” J Cutan Pathol 30(6): 363-368.
Frantz, C., K. M. Stewart, et al. (2010). “The extracellular matrix at a glance.” J Cell Sci Suppl 123(24): 4195-4200.
Fritsch, A., S. Loeckermann, et al. (2008). “A hypomorphic mouse model of dystrophic epidermolysis bullosa reveals mechanisms of disease and response to fibroblast therapy.” The Journal of Clinical Investigation 118(5): 1669-1679.
Gnanapavan, S. and G. Giovannoni (2013). “Neural cell adhesion molecules in brain plasticity and disease.” Multiple Sclerosis and Related Disorders 2(1): 13-20.
Golias, C., E. Tsoutsi, et al. (2007). “Review. Leukocyte and endothelial cell adhesion molecules in inflammation focusing on inflammatory heart disease.” In Vivo 21(5): 757-769.
Gotts, J. E. and M. A. Matthay (2012). “Mesenchymal stem cells and the stem cell niche: a new chapter.” Am J Physiol Lung Cell Mol Physiol 302(11): L1147-1149.
Halper, J. and M. Kjaer (2014). “Basic components of connective tissues and extracellular matrix: elastin, fibrillin, fibulins, fibrinogen, fibronectin, laminin, tenascins and thrombospondins.” Adv Exp Med Biol 802: 31-47.
Hellewell, P. G. (1993). “Cell adhesion molecules and potential for pharmacological intervention in lung inflammation.” Pulm Pharmacol 6(2): 109-118.
Hillis, G. S. and A. D. Flapan (1998). “Cell adhesion molecules in cardiovascular disease: a clinical perspective.” Heart 79(5): 429-431.
Jang, Y., A. M. Lincoff, et al. (1994). “Cell adhesion molecules in coronary artery disease.” J Am Coll Cardiol 24(7): 1591-1601.
Järveläinen, H., A. Sainio, et al. (2009). “Extracellular Matrix Molecules: Potential Targets in Pharmacotherapy.” Pharmacological Reviews 61(2): 198-223.
Johnson, J. P. (1991). “Cell adhesion molecules of the immunoglobulin supergene family and their role in malignant transformation and progression to metastatic disease.” Cancer Metastasis Rev 10(1): 11-22.
Johnson, J. P. (1992). “Cell adhesion molecules in neoplastic disease.” Int J Clin Lab Res 22(2): 69-72.
Kielty, C. M., M. J. Sherratt, et al. (2002). “Elastic fibres.” J Cell Sci 115(Pt 14): 2817-2828.
Kim, S. H., J. Turnbull, et al. (2011). “Extracellular matrix and cell signalling: the dynamic cooperation of integrin, proteoglycan and growth factor receptor.” J Endocrinol 209(2): 139-151.
Lian, Q., E. Lye, et al. (2007). “Derivation of clinically compliant MSCs from CD105+, CD24− differentiated human ESCs.” Stem Cells 25(2): 425-436.
Lu, P., K. Takai, et al. (2011). “Extracellular matrix degradation and remodeling in development and disease.” Cold Spring Harb Perspect Biol 3(12).
Mishra, L., B. B. Mishra, et al. (1993). “In vitro cell aggregation and cell adhesion molecules in Crohn's disease.” Gastroenterology 104(3): 772-779.
Nakamura, S., H. Ohtani, et al. (1993). “In situ expression of the cell adhesion molecules in inflammatory bowel disease. Evidence of immunologic activation of vascular endothelial cells.” Lab Invest 69(1): 77-85.
Patino, M. G., M. E. Neiders, et al. (2002). “Collagen: An Overview.” Implant Dentistry 11(3): 280-285.
Ricard-Blum, S. (2011). “The collagen family.” Cold Spring Harb Perspect Biol 3(1): a004978.
Schiller, B. and G. Elinder (1999). “Inflammatory parameters and soluble cell adhesion molecules in Swedish children with Kawasaki disease: relationship to cardiac lesions and intravenous immunoglobulin treatment.” Acta Paediatr 88(8): 844-848.
Schofield, R. (1978). “The relationship between the spleen colony-forming cell and the haemopoietic stem cell.” Blood Cells 4(1-2): 7-25.
Schuermann, G. M., A. E. Aber-Bishop, et al. (1993). “Altered expression of cell adhesion molecules in uninvolved gut in inflammatory bowel disease.” Clin Exp Immunol 94(2): 341-347.
Tkachenko, E., J. M. Rhodes, et al. (2005). “Syndecans: New Kids on the Signaling Block.” Circulation Research 96(5): 488-500.
Ukkonen, M., K. Wu, et al. (2007). “Cell surface adhesion molecules and cytokine profiles in primary progressive multiple sclerosis.” Mult Scler 13(6): 701-707.
Villanacci, V., F. Facchetti, et al. (1993). “Expression of cell adhesion molecules in jejunum biopsies of children with coeliac disease.” Ital J Gastroenterol 25(3): 109-116.
Vora, A. J., D. Kidd, et al. (1997). “Lymphocyte-endothelial cell interactions in multiple sclerosis: disease specificity and relationship to circulating tumour necrosis factor-alpha and soluble adhesion molecules.” Mult Scler 3(3): 171-179.
Wegner, C. D., R. H. Gundel, et al. (1992). “Expression and probable roles of cell adhesion molecules in lung inflammation.” Chest 101(3 Suppl): 34S-39S.

In this document and in its claims, the verb “to comprise” and its conjugations is used in its non-limiting sense to mean that items following the word are included, but items not specifically mentioned are not excluded. In addition, reference to an element by the indefinite article “a” or “an” does not exclude the possibility that more than one of the element is present, unless the context clearly requires that there be one and only one of the elements. The indefinite article “a” or “an” thus usually means “at least one”.

Each of the applications and patents mentioned in this document, and each document cited or referenced in each of the above applications and patents, including during the prosecution of each of the applications and patents (“application cited documents”) and any manufacturer's instructions or catalogues for any products cited or mentioned in each of the applications and patents and in any of the application cited documents, are hereby incorporated herein by reference. Furthermore, all documents cited in this text, and all documents cited or referenced in documents cited in this text, and any manufacturer's instructions or catalogues for any products cited or mentioned in this text, are hereby incorporated herein by reference.

Various modifications and variations of the described methods and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in molecular biology or related fields are intended to be within the scope of the claims.

1. A method of promoting, restoring or enhancing homeostasis in an individual suffering from epidermolysis bullosa (EB), the method comprising administering a therapeutically effective amount of exosomes to the individual.

2. The method according to claim 1, wherein the homeostasis comprises maintenance of an immune response.

3. The method according to claim 1, wherein the disease comprises epidermolysis bullosa (EB), epidermolysis bullosa simplex, junctional epidermolysis bullosa, dystrophic epidermolysis bullosa, lethal acantholytic epidermolysis bullosa or epidermolysis bullosa acquisita.

4. The method according to claim 1, wherein the exosomes comprise a mesenchymal stem cell exosome.

5. The method according to claim 1, wherein the exosomes comprise at least one biological property of a mesenchymal stem cell.

6. The method according to claim 1, wherein the exosomes are capable of reducing infarct size as assayed in a mouse or pig model of myocardial ischemia and reperfusion injury, or are capable of reducing oxidative stress as assayed in an in vitro assay of hydrogen peroxide (H₂O₂)-induced cell death.

7. The method according to claim 1, wherein the exosomes have a size of between 50 nm and 100 nm as determined by electron microscopy.

8. A method of treatment of an individual suffering from epidermolysis bullosa (EB), the method comprising administering an exosome to the individual.

9. The method according to claim 8, wherein the exosome is a mesenchymal stem cell exosome.

10. The method according to claim 8, wherein the disease comprises epidermolysis bullosa (EB), epidermolysis bullosa simplex, junctional epidermolysis bullosa, dystrophic epidermolysis bullosa, lethal acantholytic epidermolysis bullosa or epidermolysis bullosa acquisita.

11. The method according to claim 8, wherein the exosomes comprise at least one biological property of a mesenchymal stem cell.

12. The method according to claim 11, wherein the exosomes are capable of reducing infarct size as assayed in a mouse or pig model of myocardial ischemia and reperfusion injury, or are capable of reducing oxidative stress as assayed in an in vitro assay of hydrogen peroxide (H₂O₂)-induced cell death.

13. The method according to claim 8, wherein the exosomes have a size of between 50 nm and 100 nm as determined by electron microscopy.
